(19) United States
(12) Patent Application Publication
(10) Pub. No.: US 2022/0220180 A1
(43) Pub. Date: Jul. 14, 2022

(54) NOVEL METHOD

(71) Applicants: Babraham Institute, Cambridge (GB); VIB VZW, Gent (BE); Katholieke Universiteit Leuven, K.U. Leuven R&D, Leuven (BE)

(72) Inventors: Matthew HOLT, Cambridge (GB); Adrian LISTON, Cambridge (GB); James DOOLEY, Cambridge (GB)

(21) Appl. No.: 17/653,554

(22) Filed: Mar. 4, 2022

Related U.S. Application Data
(63) Continuation of application No. PCT/GB2020/052148, filed on Sep. 7, 2020.

(30) Foreign Application Priority Data
Sep. 6, 2019 (GB) 1912863.6

(51) Int. Cl. C07K 14/55; A61K 35/17; C12N 15/86

(52) U.S. Cl. CPC C07K 14/55; A61K 35/17; C12N 15/86; A01K 2217/052; C12N 2830/008; A01K 2227/105; A01K 2267/035; C12N 2750/14143; A01K 2217/206

(57) ABSTRACT

The invention relates to a method of expanding a population of regulatory T cells in a tissue or organ of a subject, wherein said method comprises administration of IL-2 and a targeting moiety specific for said tissue or organ, and wherein said tissue or organ is the central and/or peripheral nervous system. The invention further relates to populations of regulatory T cells produced according to the method and the production of said population in vivo. Also provided is a pharmaceutical composition comprising IL-2 and a targeting moiety as defined herein as well as a method of treating a disease or disorder mediated by inflammation or for the reduction of inflammation which comprises the methods defined herein or administration of a pharmaceutical composition as defined herein.

### FIELD OF THE INVENTION

The invention relates to a method of expanding a population of regulatory T cells in a tissue or organ of a subject, wherein said method comprises administration of IL-2 and a targeting moiety specific for said tissue or organ, and wherein said tissue or organ is the central and/or peripheral nervous system. The invention further relates to populations of regulatory T cells produced according to the method and the production of said population in vivo. Also provided is a pharmaceutical composition comprising IL-2 and a targeting moiety as defined herein as well as a method of treating a disease or disorder mediated by inflammation or for the reduction of inflammation which comprises the methods defined herein or administration of a pharmaceutical composition as defined herein.

### BACKGROUND OF THE INVENTION

Neuroinflammation is a pathogenic process in multiple neuroinflammatory diseases. As the process of inflammation is well understood, with multiple anti-inflammatory immunosuppressive drugs available, in principle neuroinflammation should be a tractable problem. The key issues preventing the use of immunosuppressive agents in neuroinflammatory diseases are: 1) the blood-brain-barrier, and 2) the issue of off-target immunosuppression. In essence, any dose of immunosuppressive agent sufficient to dampen down neuroinflammation would have to be high enough to give wide-spread peripheral immunosuppression, and as such would be untenable in patients.

Avles et al. (2017) Brain and WO 2017/060510 disclose decreased IL-2 levels in hippocampal biopsies of patients with Alzheimer's disease and describe that systemic delivery of IL-2 in a transgenic mouse model of Alzheimer's disease drives expansion and activation of systemic and brain regulatory T cells.

Dashkoff et al. (2016) Molecular Therapy describes and characterises an adeno-associated virus expressing GFP under the control of an astrocyte or neuronal promoter.

Rouse et al. (2013) Immunobiology describes the effectiveness of systemic IL-2 treatment in ameliorating pathology in a mouse model of multiple sclerosis (MS) when delivered prior to the onset of disease.

There is therefore a great need for effective treatments of inflammatory diseases or disorders.

### SUMMARY OF THE INVENTION

According to a first aspect of the invention, there is provided a method of expanding a population of regulatory T cells in a tissue or organ of a subject in need thereof, wherein said method comprises administration of IL-2 and a targeting moiety specific for said tissue or organ, and wherein said tissue or organ is the central and/or peripheral nervous system.

According to a further aspect of the invention, there is provided a pharmaceutical composition comprising IL-2 and a targeting moiety specific for a tissue or organ of a subject, wherein said targeting moiety is specific for the central and/or peripheral nervous system.

According to a yet further aspect of the invention, there is provided a method of treating a disease or disorder mediated by inflammation and/or for the reduction of inflammation, wherein said method either comprises a method as defined herein or administering to a subject in need thereof the pharmaceutical composition as defined herein.

### BRIEF DESCRIPTION OF THE FIGURES

FIG. 1: Regulatory T cells are present in the parenchyma of the healthy mouse brain.

A) Representative confocal microscopic images showing regulatory T cells, immunostained using CD4 (first column) and FoxP3 (a specific marker of regulatory T cells—second column) located in the mouse brain parenchyma, perivascular space and intravascular regions. Fluorescent-labelled lectin was used to label vasculature (third column) and cell nuclei were stained with DAPI (fourth column). Scale bar=20 μm.

B) Magnification and 3D-reconstruction of an example of CD4+Foxp3+ T cells. Scale bar=10 μm.

C) Regulatory T cells were assessed in the perfused mouse brain by high-dimensional flow cytometry. Wildtype mice were sampled during healthy aging (weeks 8, 12, 30 and 52). n=8, 5, 6 and 5 respectively.

FIG. 2: Brain-resident regulatory T cells acquire a residency phenotype in situ during a prolonged brain transit.

A) Schematic of parabiosis experiments (n=12, 12, 18, 16, 14).

B) Curve of best fit for the origin of CD4+Foxp3+ regulatory T cells in the blood and brain, showing the CD69+ population in the brain.

C) tSNE of CD4+Foxp3+ regulatory T cells gated on CD4+Foxp3−CD3+CD8−CD45+, built on CD62L, CD44, CD103, CD69, CD25, PD-1, Nrp1, ICOS, KLRG1, ST2, Ki67, Helios, T-bet and CTLA4. CD69 expression is shown in grayscale. Host and incoming cells were defined on CD45.1 vs CD45.2 expression, and are shown at the 2, 4 and 8 week timepoints.

D) CD69 histograms for CD4+Foxp3+ regulatory T cells. Host and incoming cells were defined on CD45.1 vs CD45.2 expression, and are shown at the 2, 4 and 8 week timepoints.

E) Population flow diagrams for CD4+Foxp3+ regulatory T cells, in homeostatic state. Circle areas represent population frequencies, calculated independently for blood and brain. Small black circles represent cell death. The size of arrow ends is proportional to the rate of population flow, as exit (outgoing arrow) or entry (incoming arrow). All sizes of arrows ends are equally scaled in each panel, so that the population with highest turnover has arrows covering the complete circumference (thus, this graphical representation of population flow is the same irrespective of the unit used for transition rates). Numbers close to each arrow end display the corresponding entry or exit rate, in events/1000 cells/day. Numbers with asterisk denote rates with high estimation uncertainty. Population transitions with rates lower than 0.1/1000 cells/day at both ends are not shown.

FIG. 3: Transgenic mouse model for proof-of-principle brain-specific regulatory T cell expansion.

A) The Rosa^{fl-Stop-fl}IL-2 allele contains a floxed stop cassette, IL-2 expression is activated after Cre activity. Using a CD4Cre driver we compared the transgene-induced level of IL-2 production to the endogenous stimulation-induced level of IL-2 reduction.

B) and C) Schematic of tamoxifen inducible Cre (Cre^{ERT2}) under control of the brain-specific promoters tested in this study: B) Plp1 and C) CaMKII.

D) Effect of brain specific IL-2 production on regulatory T cell population expansion proliferation. Plots comparing Treg (Foxp3+CD25+) expansion in blood and brain in wildtype, IL-2 Plp1Cre and IL-2 aCaMKII Cre mice.

E) Histograms showing the percentages of Foxp3+ cells in the CD4+ cell population. Mean±SEM (P value, One Way Anova).

F) 10× Chromium single cell sequencing was performed on CD4 T cells from the wildtype perfused adult IL-2 aCaMKII Cre mouse brain. tSNE visualising cell clusters built on the combined population. Clusters of naïve CD4 T cells, activated CD4 T cells and CD4+Foxp3+ regulatory T cells are identified and labelled (top) based on signature expression of transcriptional markers (bottom).

G) Fold-change of all expressed genes between conventional T cells and regulatory T cells of IL-2 aCaMKII Cre mice.

H) Transcription profile of cytokines in CD4+ T cells purified from the murine IL-2 aCaMKII Cre brain, with analysis through the 10× single cell pipeline.

I) to S) Behavioral assessment of IL-2 aCaMKII Cre (αCamKII^{IL2}) and control mice. I) Time spent on the rod, average of 4 repeated tests of 300 seconds (n=23, 17). J) Open field, total distance moved and K) time in the corners (n=23, 16). L) Nest building scoring (n=24, 18). M) Light-dark test latency to enter light zones and N) time spent in the light zone in (n=20, 17). O) Time immobile during forced swim test (n=24, 16). P) Sociability test trials to monitor the interaction with a stranger mouse (S) compared to an empty chamber (E) (n=28, 18). Q) Freezing behaviour over time during context acquisition conditioning (n=28, 18). Mean±SEM. R) Contextual discrimination during generalization test. Mean±SEM (n=28, 18. S) Spatial learning in the Morris water maze. Path length to finding the hidden platform (n=16, 8), probe tests after 5 days and 10 days and after reversal learning (n=28, 20). Mean±SEM.

FIG. 4: Expanded brain regulatory T cells protect against traumatic brain injury.

Wildtype littermates and IL-2 aCaMKII Cre (αCamKII^{IL2}) mice were given controlled cortical impacts to induce moderate traumatic brain injury (TBI) and examined at 15 days post-TBI.

A) Macroscopic damage to the surface of the brain at the injury site.

B) Representative confocal images captured within the brain of IL-2 aCaMKII Cre (αCamKII^{IL2}) or littermate control mouse 15 days following cortical injury on the ipsilateral side.

C) Immunofluorescence staining of the cortical tissue after controlled cortical impact surgery. GFAP (astrocytes), NeuN (neurons), DAPI (nuclei). Scale bars=50 μm.

D) Lesioned area, shown as percentage of the entire hemisphere (n=3, 3).

FIG. 5: Astrocyte specific expression using a GFAP promoter.

A) The GFAP promoter restricts expression of TdTomato to astrocytes in adult mouse brain, as judged by characteristic cell morphology and by immunostaining for the astrocyte specific markers, GFAP and S100β. Off-target expression was not detected when slices were counter-stained for NeuN (neurons), APC (oligodendrocytes), IBA1 (microglia), and PDGFRa (NG2+ cells). Scale bars=20 μm. Data are representative images seen in 3 slices from 3 independent mice receiving the GFAP-TdTomato construct.

B) Representative staining (left) and quantified expression (right) of GFAP in the cortex and striatum of adult mouse brain, 14 days post-induction of traumatic brain injury (TBI; n=5), with quantification.

FIG. 6: PHP.B-GFAP-IL2 specifically expands brain Tregs and controls neuroinflammation.

A) Flow cytometric analysis of cells isolated from brain of C571Bl6 mice infected with PHP.B control (PHP.B-GFP) or PHP.B-GFAP-IL2. Cells were gated on live CD45+CD11b−CD19−CD3+.

B) Frequency of Tregs (CD4+Foxp3+ cells) in the brain. The data are shown as mean±SEM (n=3 per group).

C) Flow cytometric analysis of cells isolated from spleen of C57Bl6 mice infected with PHP.B control (PHP.B-GFP) or PHP.B-GFAP-IL2.

D) Frequency of regulatory T cells in the spleen. The data are shown as mean±SEM (n=3 per group).

E) Blood, spleen and perfused mouse brain from PHP.B-GFAP-GFP control and PHP.B-GFAP-IL2-treated mice were compared by high-dimensional flow cytometry for regulatory T cell numbers.

F) Wildtype mice were administered 10⁹, 10¹⁰ or 10¹¹ vector genomes (total dose) of PHP.B-GFP control vector or PHP.B-GFAP-IL2 by tail vein injection and assessed for the number of conventional (left) and regulatory (right) T cells in the perfused brain 14 days after treatment (n=3-5 per group).

G) C57Bl6 mice infected with PHP.B-GFP control or PHP.B-GFAP-IL2 (10⁹ vg/mouse). 14 days after the infection with PHP.B, mice were immunized with MOG⁽³⁵⁻⁵⁵⁾ in CFA to induce EAE. Mononuclear cells were isolated at day 30 of EAE. Clinical scores and mean with SEM of cumulative clinical scores were calculated. (n=15, 14; mean±SEM; P value, Mann-Whitney U test).

H) Cells were isolated from CNS (brain and spinal cord). Top row: absolute numbers or frequency of the indicated brain-infiltrating cells are shown. Bottom row: CNS-derived cells were stimulated with PMA and ionomycin to analyse IL-10, IL-17, GM-CSF, and Amphiregulin (AREG) in CD4 or regulatory T cells by flow cytometry. Symbols depict individual mice. The data are shown as mean±SEM (n=6-7 per group).

I) As in G) but with mice treated with PHP.B-GFAP-IL2 or PHP.B-GFP control 10 days after induction of EAE (indicated by arrow). Incidence, daily clinical score (mean±SEM) and cumulative mean clinical score (n=15, 14).

FIG. 7: PHP.B-GFAP-IL2 protects against traumatic brain injury.

Mice were injected i.v. with 1× dose of 1×10⁹ vector genomes per mouse of PHP.B-GFAP-IL2 or PHP.B control (PHP.B-GFP) at −14 days prior to controlled cortical impacts to induce moderate traumatic brain injury (TBI). Brains of mice were examined at 15 days post-TBI.

A) Macroscopic damage to the surface of the brain at the injury site.

B) Representative confocal images captured within the brain of control PHP.B-GFP, PHP.B-GFAP-IL2 or sham surgery mice following cortical injury on the ipsilateral side, showing NeuN, BrdU and GFAP.

C) Quantification of area of lesion lost, relative Iba1 expression in the cortex and striatum and GFAP expression in the cortex and striatum (ratio of expression in ipsilateral vs. contralateral hemispheres).

D) Representative MRI and MRI-based quantification of lesion size, in PHP.B-GFAP-GFP control or PHP.B-GFAP-IL2-treated mice on days 1, 7, 14, 35 and 150 post-TBI (control n=16, 16, 12, 11, 10; IL2 n=16, 16, 16, 12, 9).

E) Percentage of total time spent in the target quadrant during the probe trial.

F) Ratio of exploration time of novel over old object during day 2 of the Novel Object Recognition paradigm.

FIG. 8: Normal peripheral influx following PHP.B-GFAP-IL2 treatment in traumatic brain injury mice.

Mice, treated day −14 with PHP.B-GFAP-IL2 or control PHP.B-GFAP-GFP were given controlled cortical impacts to induce moderate traumatic brain injury (TBI) and examined at 15 days post-TBI (n=3, 4, 4), a sham TBI was included in the control PHP.B-GFAP-GFP group. TBI-induced perfused brains from sham, TBI and PHP.B-GFAP-IL2-treated TBI mice were compared by high-dimensional flow cytometry.

A) Microglia, gated on CD11b⁺ CX3CR1⁺ CD64⁺ CD45^{mod} Ly6G⁻ cells, as a proportion of CD45⁺ cells or B) absolute number.

C) Expression of MHCII on microglia.

D) Percentage of CD4 and CD8 T cells, as a proportion of CD45⁺ CD11b⁻ TCRβ⁺ CD19⁻ cells.

E) Percentage of regulatory T cells (CD4⁺ Foxp3⁺) as a proportion of CD4 T cells.

F) Frequency of CD25, CD44, CD69, Ki67 and PDL1 expressing-cells.

G) Frequency or H) mean fluorescence intensity (MFI) of Amphiregulin-producing cells, within the CD4 conventional T cell population.

I) Frequency of CD25, CD44, CD69, Ki67 and PDL1 expressing-cells.

J) Frequency or (K) mean expression of Amphiregulin-producing cells, within the CD4 conventional T cell population. Mean±SEM.

FIG. 9: Expansion of Regulatory T cells in the Brain Reduces Severity in Stroke.

A) Wildtype mice, treated with control PHP.B-GFAP-GFP or PHP.B-GFAP-IL2 on day −14 (n=7, 10), were given a distal middle artery occlusion (dMCAO) stroke and examined at 15 days post-stroke for macroscopic damage and B) TTC-based quantification of damage.

C) Wildtype mice, treated with control PHP.B-GFAP-GFP or PHP.B-GFAP-IL2 on day −14 (n=5, 5), were given a photothrombotic stroke and examined one day post-stroke for macroscopic damage, with representative images and D) TTC-based quantification.

FIG. 10: A Small-Molecule Inducible System for Brain-Specific Regulatory T cell Expansion.

Wildtype mice were administered 10⁹ vector genomes (total dose) of PHP.B-GFAP-GFP control vector or PHP.B-GFAP-TetR-T2A-rtTA(V7/V14).TetO-IL2 (PHP.GFAP/TetO-IL2) by tail vein injection. Mice were gavaged daily with minomycin (50 mg/kg) or PBS control (n=4-5 mice/group) then assessed for the proportion of Tregs in the spleen or perfused brain 14 days after treatment.

### DETAILED DESCRIPTION OF THE INVENTION

According to a first aspect of the invention, there is provided a method of expanding a population of regulatory T cells in a tissue or organ of a subject in need thereof, wherein said method comprises administration of IL-2 and a targeting moiety specific for said tissue or organ, and wherein said tissue or organ is the central and/or peripheral nervous system.

In one embodiment, the methods defined herein comprise expanding a population of cells, such as a population of regulatory T cells. In a further embodiment, said expanding of a population of cells, such as a population of regulatory T cells, is in a tissue or organ of a subject in need thereof, such as a particular tissue or organ of interest.

References herein to the terms “expanding”, “expansion” and “expanded” or to the phrases “expanding a population of regulatory T cells” and “expanded population of regulatory T cells” include references to populations of cells which are larger than or comprise a larger number of cells than a non-expanded population. It will thus be appreciated that such an “expanded” population produced according to the methods defined herein comprises a larger number of cells than a population which has not been subjected to IL-2. Thus, in certain embodiments, the expanded population of cells produced according to the methods defined herein, such as an expanded population of regulatory T cells, comprises a larger number of cells compared to a reference population of cells. In one embodiment, the reference population of cells may be a population of cells not subjected to or administered with IL-2. In one embodiment, the expanded population of cells produced according to the methods defined herein, such as an expanded population of regulatory T cells, comprises a larger number of cells than the population prior to any administration of IL-2. In further embodiments, the reference population of cells may be located in a different tissue or organ to the expanded population of cells produced according to the methods defined herein. In a further embodiment, the expanded population of cells produced according to the methods defined herein, such as an expanded population of regulatory T cells, is an expanded population in a tissue or organ of a subject and comprises a larger number of cells compared to a population of cells not located in said tissue or organ of interest. In a further embodiment, the expanded population of cells produced according to the methods defined herein, such as an expanded population of regulatory T cells, is located in a tissue or organ separated from other tissues or organs by a barrier (such as the blood-brain barrier) and comprises a larger number of cells compared to a population of cells not located with said barrier-separated tissue or organ.

In one embodiment, the expanded population of cells produced according to the methods defined herein, such as an expanded population of regulatory T cells, comprises a population at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold or more larger than a population of cells which has not been subjected to or administered with IL-2. In a further embodiment, the expanded population of cells produced according to the methods defined herein, such as an expanded population of regulatory T cells, comprises a population at least 2-fold, at least 3-fold, at least 4-fold, at least 5-fold, at least 6-fold, at least 7-fold, at least 8-fold, at least 9-fold, at least 10-fold, at least 11-fold, at least 12-fold, at least 13-fold, at least 14-fold or more larger than a population of cells not located in the tissue or organ of interest. In a particular embodiment, the expanded population of cells produced according to the methods defined herein is at least 2-fold, at least 4-fold, at least 5-fold, at least 6-fold, at least 7-fold, at least 8-fold, at least 12-fold, at least 13-fold or at least 14-fold larger than a reference population, such as a population of cells in the tissue or organ of interest which has not been subjected to or administered with IL-2 or a population of cells not located in the tissue or organ of interest. In some embodiments, the expanded population of cells produced according to the methods defined herein, such as an expanded population of regulatory T cells, comprises a larger proportion of cells which make up a subset of the population (e.g. a larger proportion of regulatory T cells within the total population of T cells in the tissue or organ).

Therefore, it will be appreciated that the expanded population of regulatory T cells as defined herein may be expanded in a manner which is dependent on the dose of IL-2 administered. Thus in certain embodiments, the expanded population of regulatory T cells as defined herein comprises a population which is larger than a reference population by a factor which is IL-2 dose-dependent.

In further embodiments, the expanded population of regulatory T cells produced according to the methods defined herein comprises a population of cells which have increased survival. Thus, in one embodiment, the expanded population of regulatory T cells produced according to the methods defined herein comprises increased survival. In a further embodiment, the expanded population of regulatory T cells produced according to the methods defined herein comprises decreased, or reduced, cell death. In a yet further embodiment, the expanded population of regulatory T cells comprise increased proliferation. Thus, in one embodiment, the expanded population of regulatory T cells produced according to the methods defined herein is larger than a reference population (e.g. a population of regulatory T cells not subjected to or administered with IL-2 or a population of cells not located in the tissue or organ of interest) because of increased survival of the expanded population of regulatory T cells. In a further embodiment, the expanded population of regulatory T cells produced according to the methods defined herein is larger than a reference population because of decreased, or reduced, cell death in the expanded population of regulatory T cells. In a yet further embodiment, the expanded population of regulatory T cells is larger than a reference population because of increased proliferation. In a still further embodiment, the expanded population of regulatory T cells produced according to the methods defined herein is larger than a reference population because of a combination of one or more of increased survival, decreased/reduced cell death and increased proliferation.

It will be appreciated that references herein to an “expanded population” produced according to the methods defined herein, such as an “expanded population of regulatory T cells”, may also include a population of cells which are activated. References herein to “expanding” may include the activation of a population of cells produced according to the methods defined herein, such as a population of regulatory T cells. Similarly, “expanding” also includes the expansion of an activated population of regulatory T cells, for example, a population which is already activated prior to administration of IL-2. Such activation of the population of cells produced according to the methods defined herein, such as a population of regulatory T cells, may be independent of an expansion or may be concomitant with an expansion of said population. Thus, in one embodiment, the expanded population of regulatory T cells produced according to the methods defined herein comprises activated regulatory T cells. In a further embodiment, the expanded population of regulatory T cells produced according to the methods defined herein is an activated population of regulatory T cells.

In an alternative embodiment, references herein to “expanding” or an “expanded population” produced according to the methods defined herein do not include activating said population or an activated population of cells. Thus, according to this embodiment, the expanded population of cells produced according to the methods defined herein, such as an expanded population of regulatory T cells, does not comprise an activated phenotype. In a further embodiment, the expanded population of regulatory T cells produced according to the methods defined herein does not comprise activated regulatory T cells. Thus, in a yet further embodiment, the expanded population of regulatory T cells produced according to the methods defined herein comprises the phenotype, such as the surface phenotype, of a population of regulatory T cells which have not been subjected to or administered with IL-2.

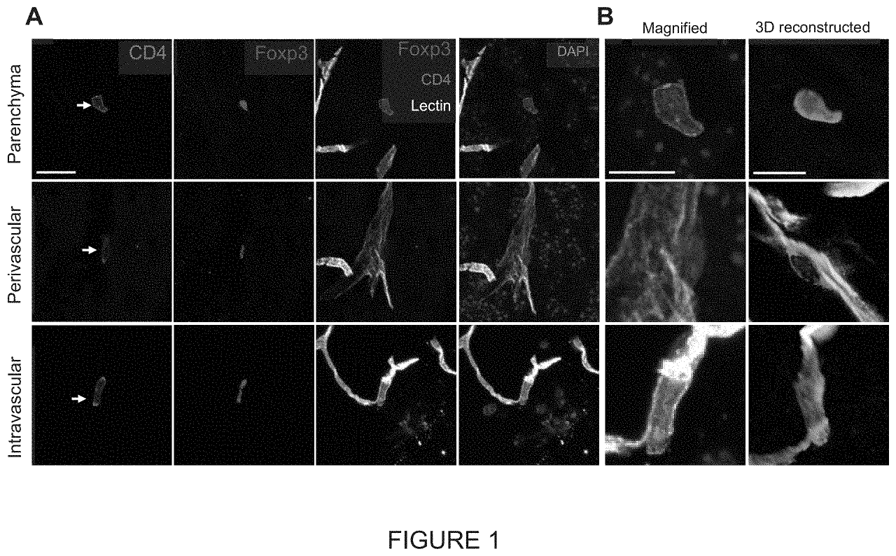

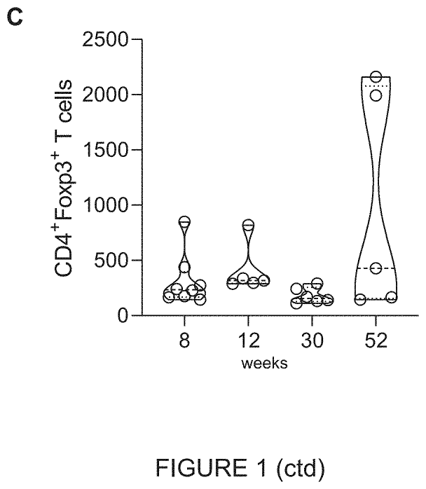

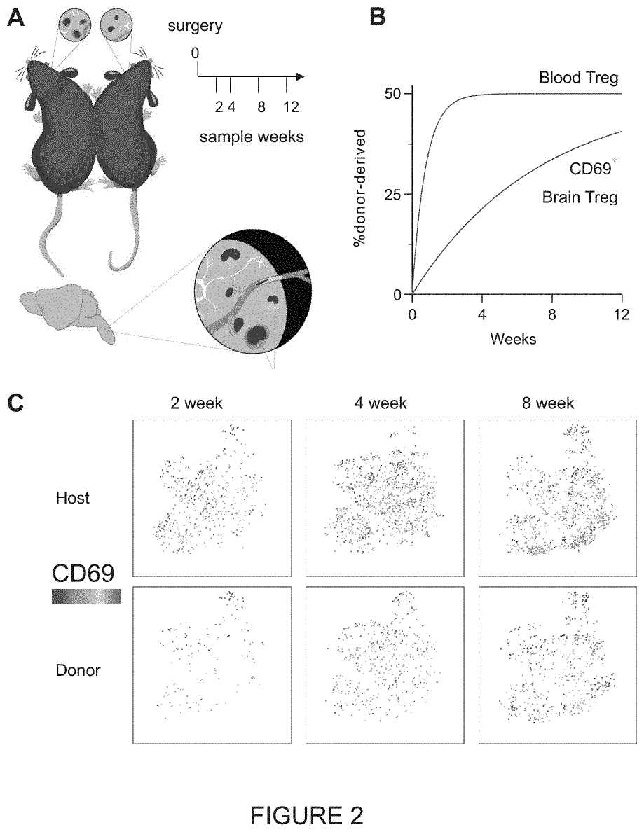

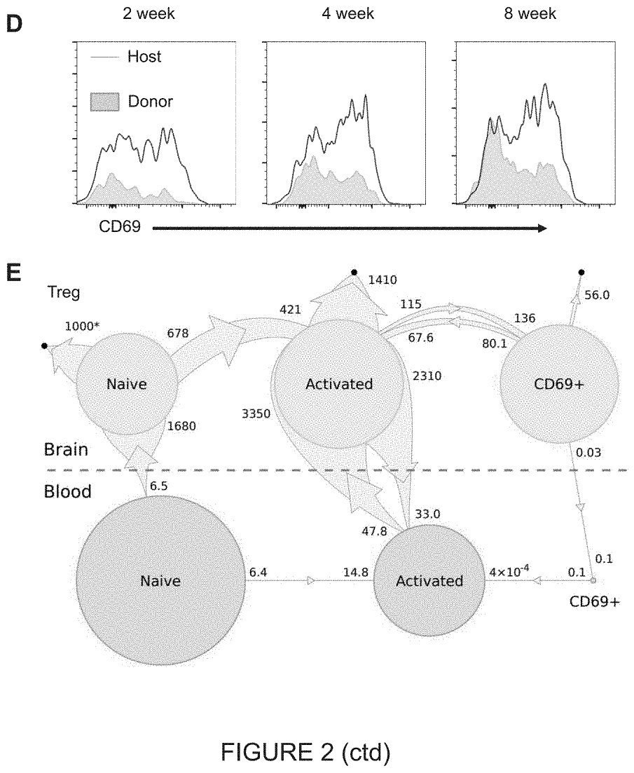

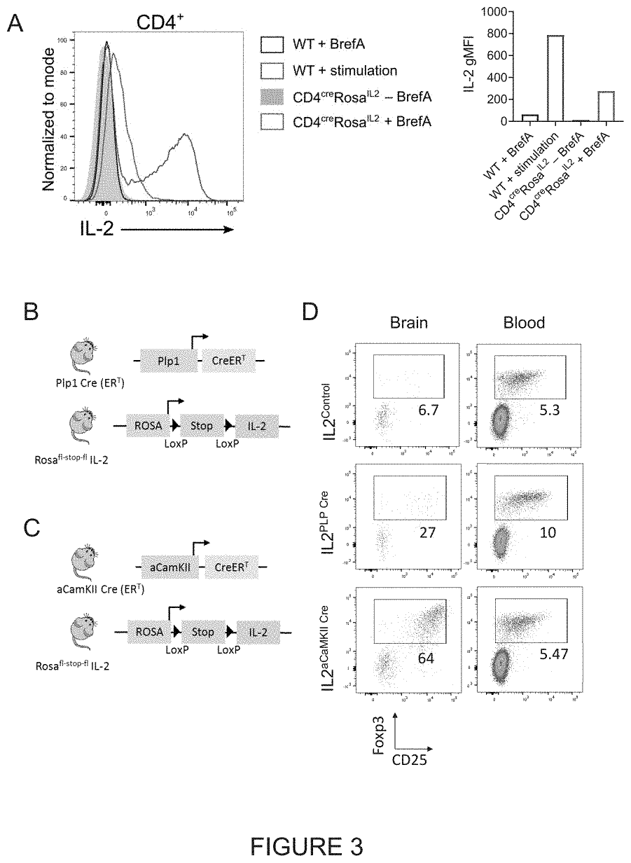

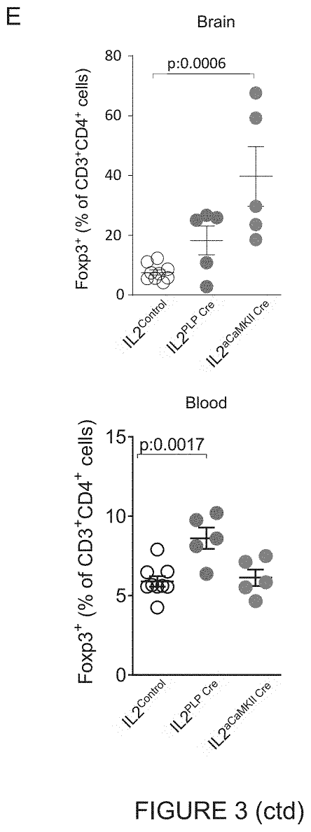

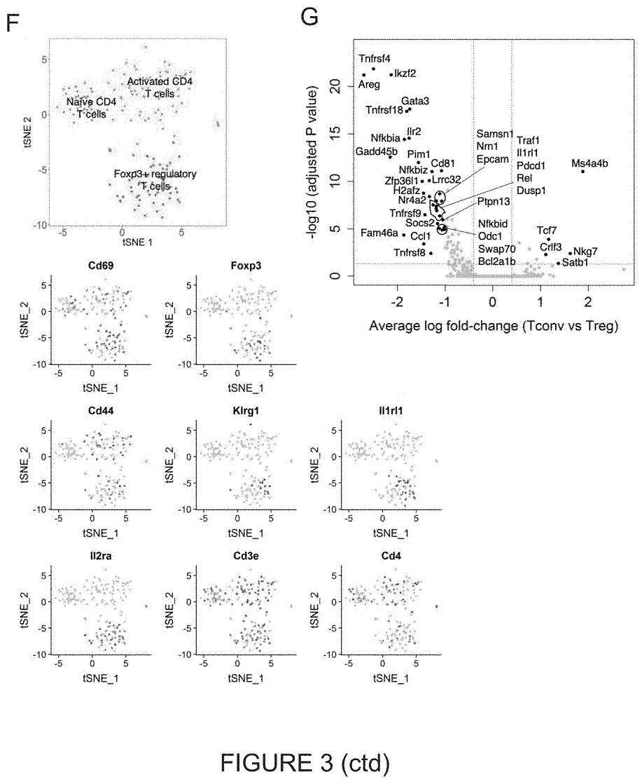

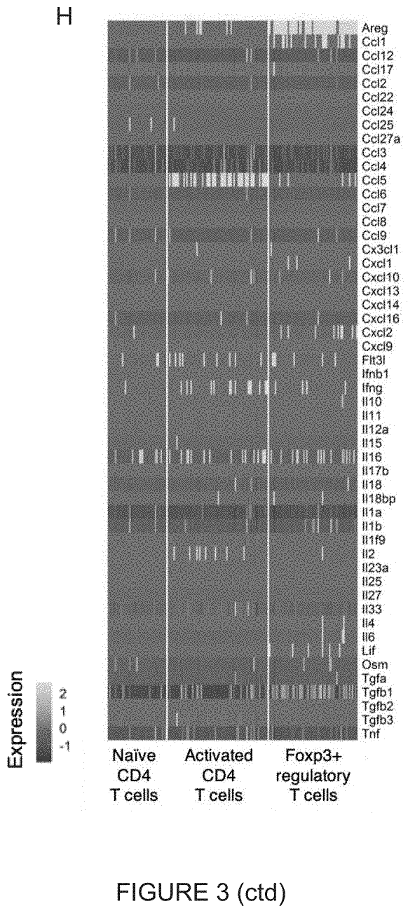

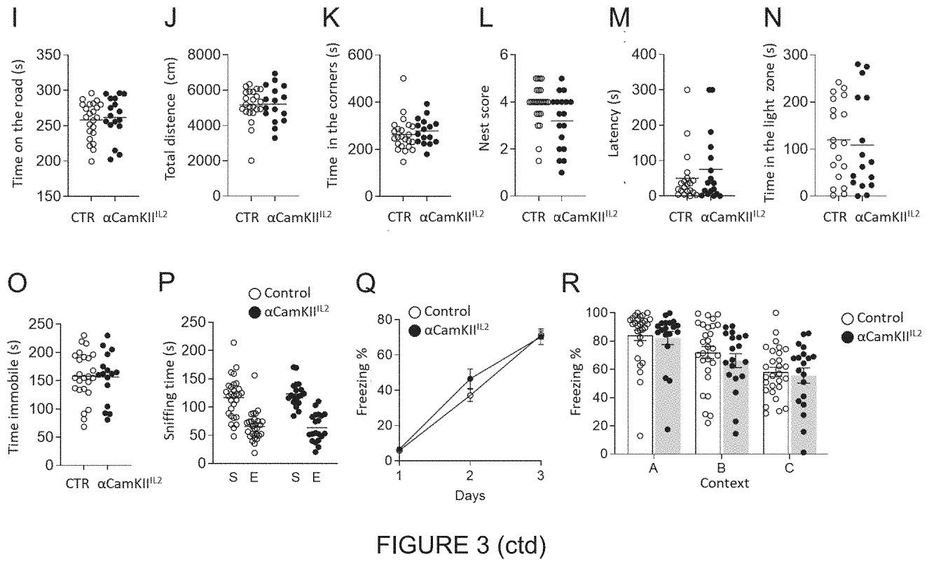

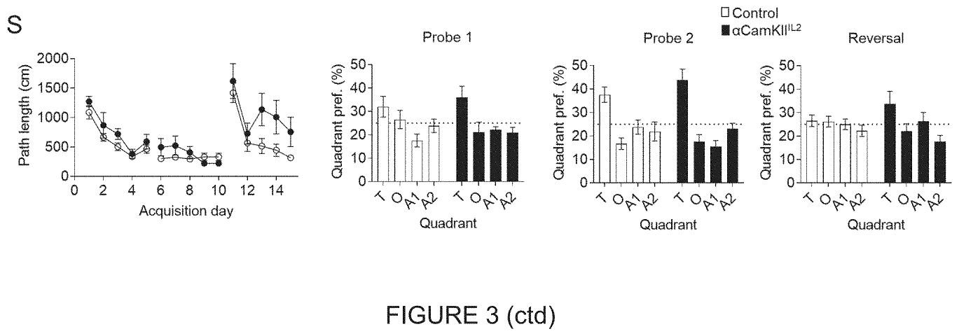

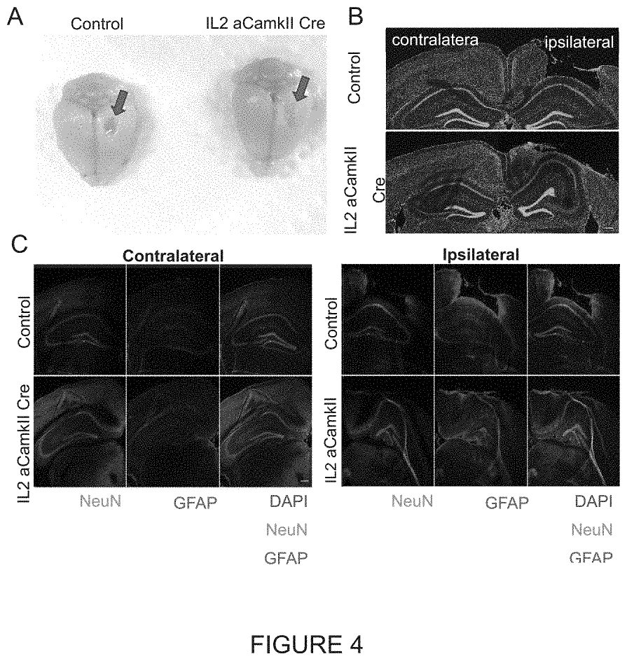

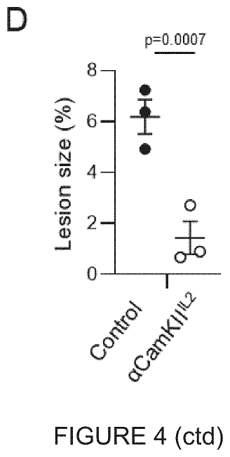

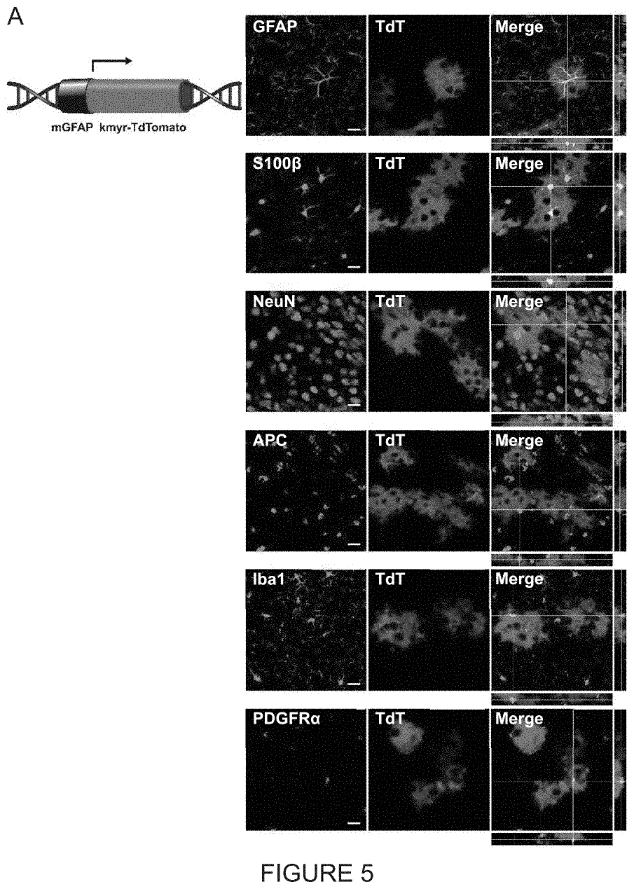

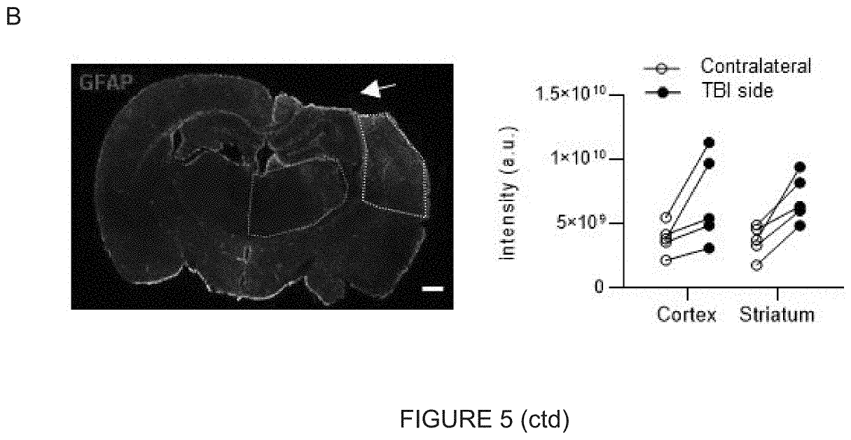

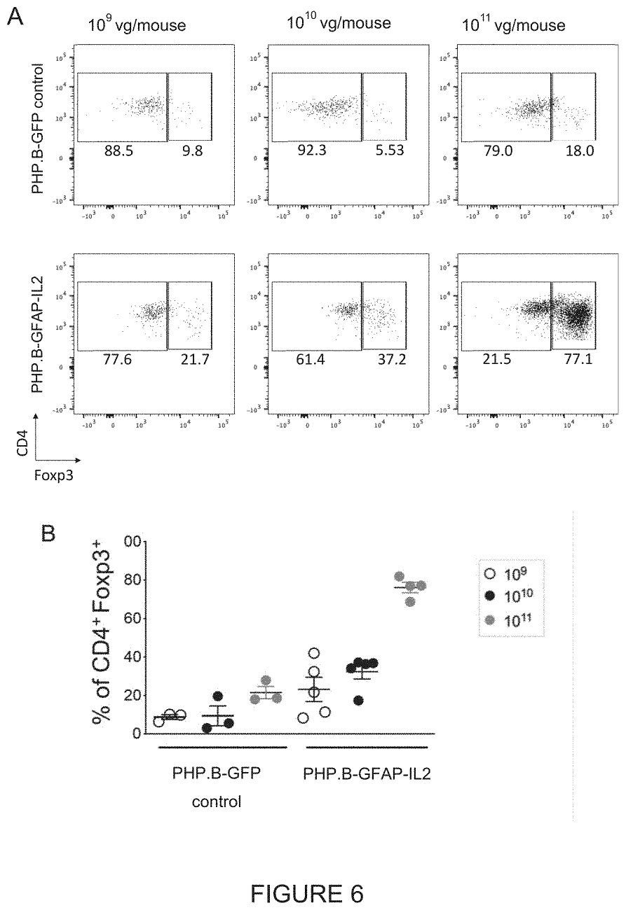

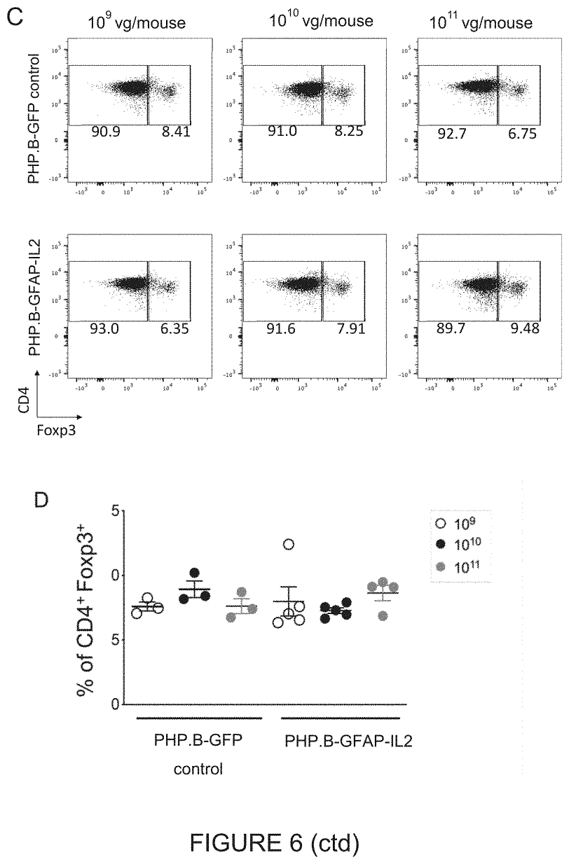

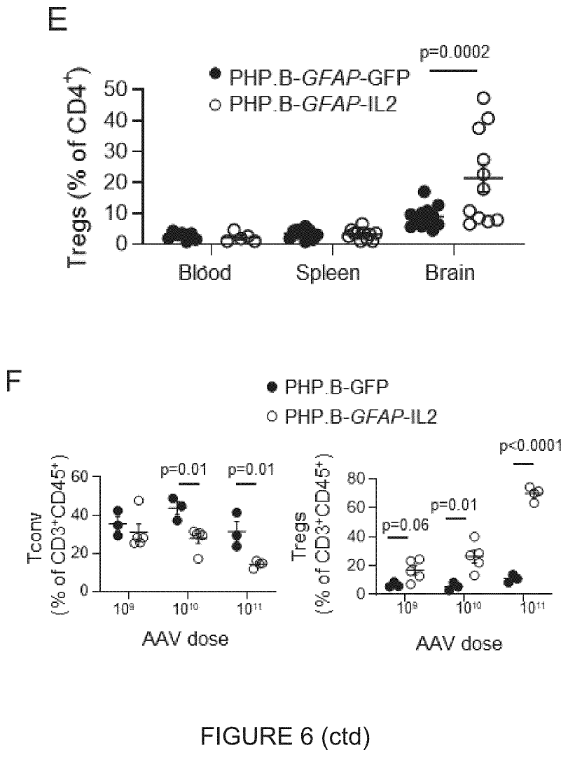

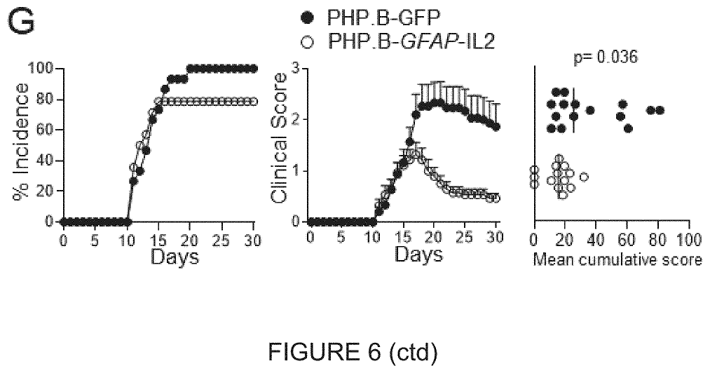

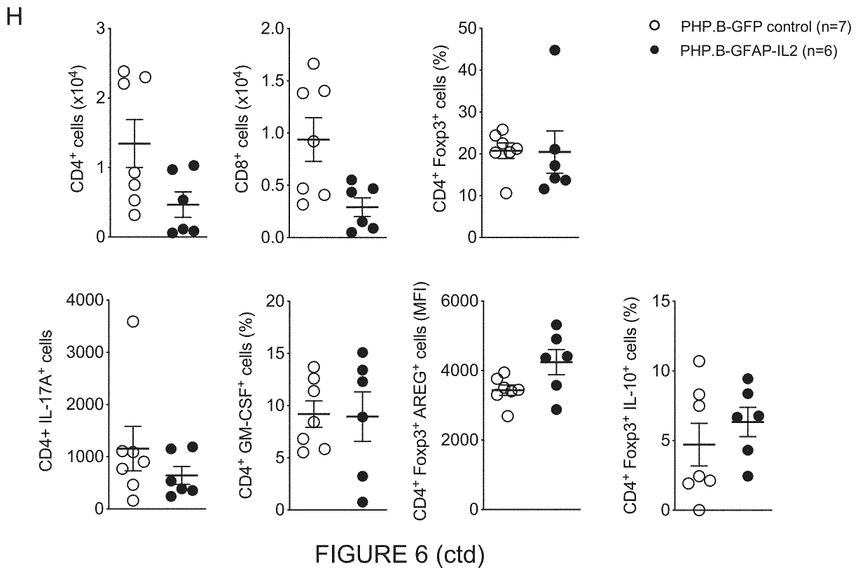

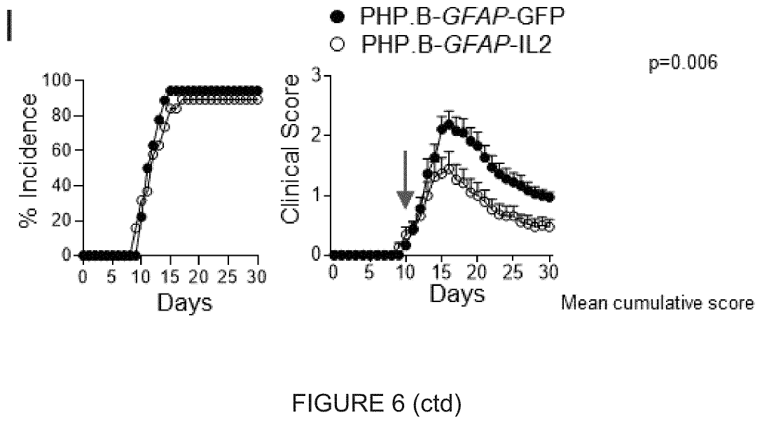

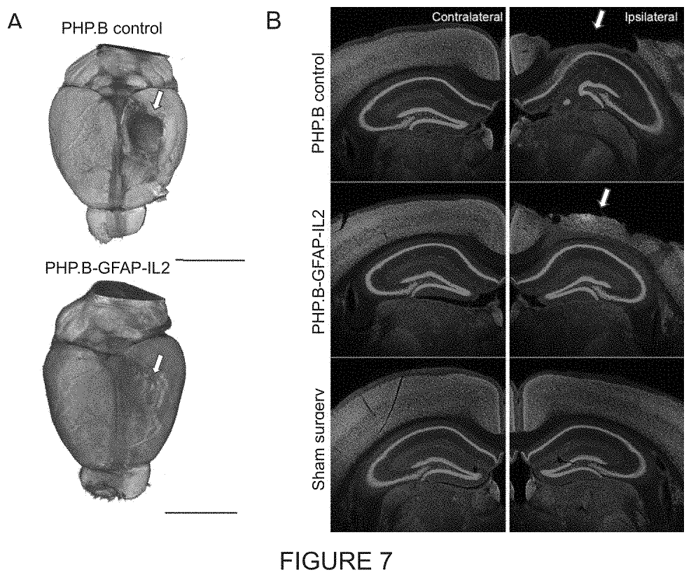

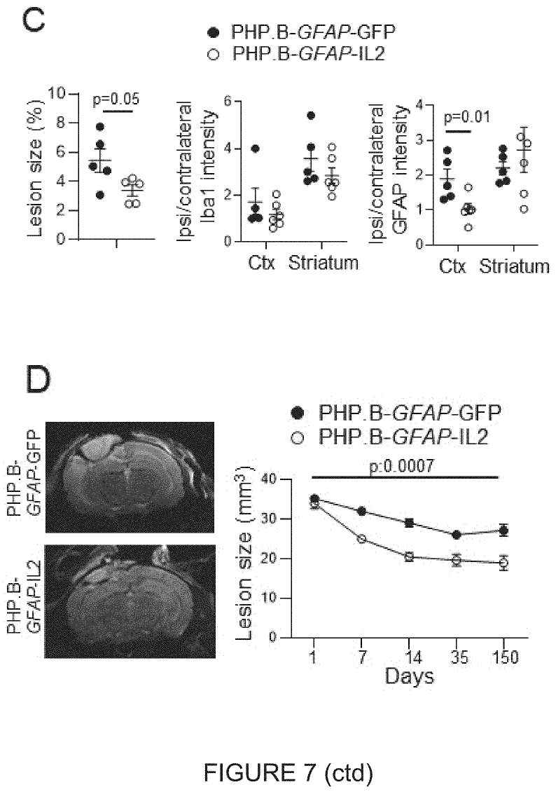

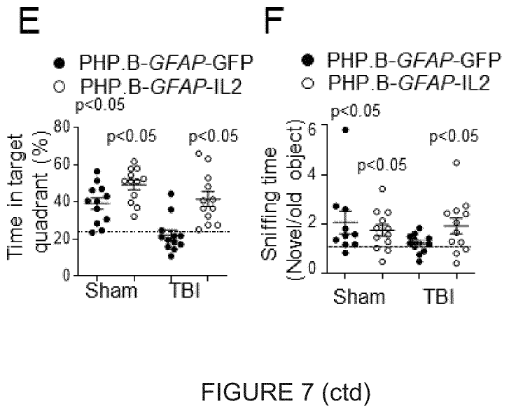

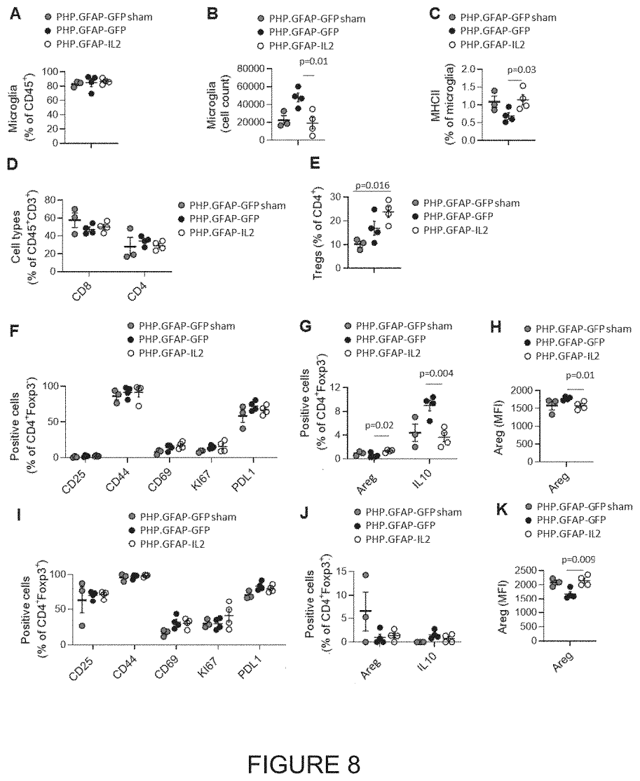

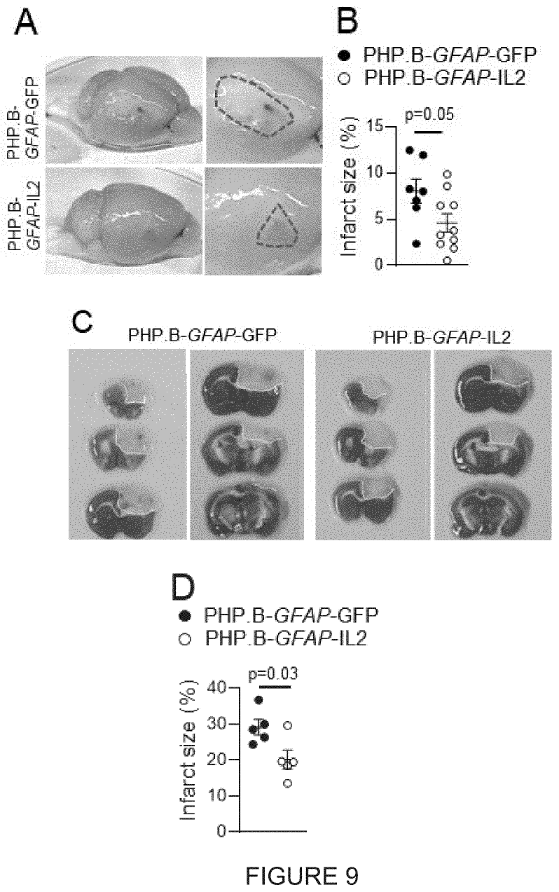

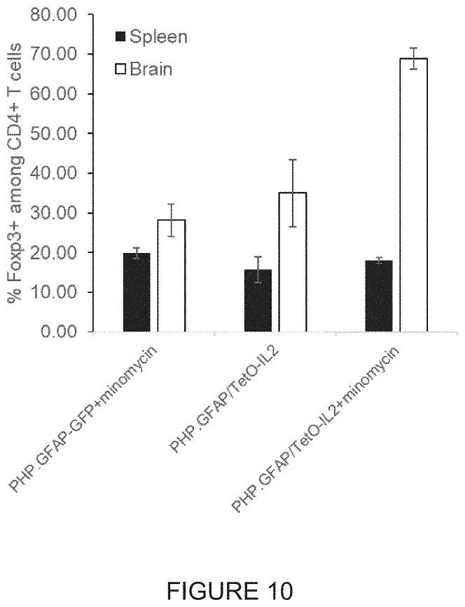

Regulatory T cells (also known as Tregs) are a subpopulation of T cells that modulate the immune system, maintain tolerance and prevent autoimmune disease. They generally suppress or downregulate the activation and/or proliferation of effector T cells and have been shown to have utility in immunosuppression. As such, regulatory T cells are highly potent cells that combine multiple immunosuppressive and regenerative capabilities and there is great interest in using exogenous regulatory T cells as a cell therapy or exogenous factors which stimulate, activate or expand endogenous regulatory T cells. The present inventors have demonstrated that regulatory T cells exist in the healthy brain (FIG. 1), despite the traditional view that the brain is a tissue which is isolated from the immune system (e.g. because of the blood-brain barrier), and thus may be a valid target for immunosuppressive treatment, such as anti-inflammatory treatment, in the brain.

Thus, in one embodiment, the expanded population of regulatory T cells produced according to the methods defined herein comprises an increased anti-inflammatory potential. Such increased anti-inflammatory potential may be compared to a non-expanded population of regulatory T cells, such as a non-expanded population of regulatory T cells present in the tissue or organ, or to a population of regulatory T cells present at another location other than the tissue or organ of interest. In one embodiment, the expanded population of regulatory T cells produced according to the methods defined herein comprises a phenotype similar to non-expanded regulatory T cells within the tissue or organ of interest or to regulatory T cells from a location other than the tissue or organ of interest. Such phenotypes may include surface marker phenotype, transcriptomic phenotype/signature (e.g. gene expression signature), gene and/or protein expression profile and cytokine expression profile. Thus, in a particular embodiment, the expanded population of regulatory T cells produced according to the methods defined herein comprises or retains the anti-inflammatory potential of a non-expanded population of regulatory T cells or the expanded population of regulatory T cells prior to expansion. In a further embodiment, the expanded population of regulatory T cells produced according to the methods defined herein comprises or retains the anti-inflammatory potential of a population of regulatory T cells from another location other than the tissue or organ of interest.

References herein to the phrase “in a tissue or organ” refer to a discrete location in the subject such as in a particular tissue or organ. It will be appreciated that such terms do not relate to wherein an effect is produced systemically or outside of the tissue or organ of interest, or wherein a cell type or cell population not located in the tissue or organ of interest is affected (e.g. expanded or activated). Thus, in one embodiment the population of regulatory T cells produced according to the methods defined herein is affected (e.g. expanded) in a particular tissue or organ, i.e. locally. In a further embodiment, the population of regulatory T cells produced according to the methods defined herein is affected (e.g. expanded) in a particular tissue or organ only. In a yet further embodiment, the population of regulatory T cells located outside or not in the tissue or organ of interest is not affected (e.g. expanded). Thus, in particular embodiments, the systemic or peripheral population of regulatory T cells is not affected (e.g. expanded).

Tissues or organs as defined herein comprise a discrete location of the body or of an organism. For example, the tissue or organ may comprise a compartment of the body such as the nervous system (e.g. the central or peripheral nervous system or the brain). In a particular embodiment, the tissue or organ is separated from other tissues or organs by a barrier, such as the blood-brain barrier. Thus, in one embodiment, the tissue or organ is the central and/or peripheral nervous system. In a further embodiment, the tissue or organ is the brain.

IL-2 is a key population control factor for regulatory T cells. Regulatory T cells have a naturally high turnover frequency compared to other T cells, with rapid proliferation and high apoptosis rates. IL-2 is able to increase the frequency of regulatory T cells through the induction of the anti-apoptotic protein Mcl1, which in turn reduces the Bim-dependent apoptotic rate (Pierson et al. (2013), doi: http://doi.org/10.1038/ni.2649). Increased IL-2 levels can therefore expand the size of the regulatory T cell population (Liston and Gray (2014), doi: https://doi.org/10.038/nri3605). IL-2 delivery has been shown to be a potent anti-inflammatory agent via the expansion of this regulatory T cell population in multiple pre-clinical studies, and optimisation of IL-2 delivery is being clinically investigated. Therefore, in the context of the brain, for the potential use of IL-2 as an anti-inflammatory mediator, the systemic delivery of IL-2 should, in theory, drive an increase in regulatory T cell numbers in the brain as this population is seeded by regulatory T cells in the circulation (FIG. 2).

In practice, however, systemic expansion of regulatory T cells through provision of IL-2 disproportionately increases the naïve regulatory T cell population which seeds the brain at approximately 10-fold lower levels of efficiency (see FIG. 2E). Therefore, the levels of systemic IL-2 provision that create a substantial increase in anti-inflammatory potential in the periphery do not create notable increases in regulatory T cell numbers in the brain. This finding presented herein indicates that while IL-2 has a high potential as a therapeutic for inflammation in the brain, such as neuroinflammation, systemic delivery in the physiological range required to boost brain regulatory T cell numbers is highly likely to induce systemic immunosuppression. By contrast, brain-specific expansion or increase in regulatory T cell numbers could induce the anti-inflammatory properties of regulatory T cells locally, without the detrimental effects of systemic immunosuppression.

Thus, according to certain embodiments of the present invention, there is provided herein a method of expanding a population of regulatory T cells in a tissue or organ of a subject in need thereof, wherein the tissue or organ is separated from other tissues or organs by a barrier, such as the blood-brain barrier. It will therefore be appreciated that the methods defined herein provide for the expansion of a population of regulatory T cells within a tissue or organ which, due to the presence of a barrier such as the blood-brain barrier, is difficult to achieve with systemic delivery of IL-2. For example, due to the presence of said barrier any dose of IL-2 sufficient to affect a population of cells present in the tissue or organ would have to be at a level high enough to give wide-spread peripheral or systemic effects. In the case of a population of regulatory T cells expanded in a tissue or organ using IL-2 as described herein, the resulting wide-spread peripheral or systemic immunosuppression would be untenable to patients due to an increased risk of infection.

References herein to “administration” will be appreciated to refer to the providing or the making available of IL-2 at a discrete location or site of the organism, such as a particular tissue or organ. Such administration will therefore be likened with the definitions of “in a tissue or organ” as previously described herein. Thus, in one embodiment, administration of IL-2 comprises administration to or in a particular tissue or organ. In particular embodiments, administration of IL-2 comprises expression of IL-2 in a particular tissue or organ (e.g. the brain or nervous system). In one embodiment, administration comprises expression of a gene encoding for IL-2 in a particular tissue or organ (e.g. the brain or nervous system). In a further embodiment, expression of IL-2 is not detectable outside the tissue or organ of interest, such as in the periphery. In a yet further embodiment, expression of IL-2 is expression which is restricted to the particular tissue or organ of interest. In a further embodiment, expression of IL-2 is tissue- or organ-specific expression. In certain embodiments, administration or expression of IL-2 may be in more than one tissue or organ of interest. In one embodiment, administration or expression of IL-2 is in one, two, or more related tissues or organs (e.g. in the brain and nervous system or in tissues of the intestinal tract). In another embodiment, administration or expression of IL-2 is in one, two, or more tissues or organs considered not to be related.

Furthermore, references herein to “administration” and “expression” also refer to wherein IL-2 is provided to a population of cells in a tissue or organ. Such provision of IL-2 may, in one embodiment, comprise administration of IL-2 in protein or peptide form to or in the tissue or organ of interest, i.e. locally. In a further embodiment, the provision of IL-2 comprises the expression of IL-2 in the cells of the tissue or organ of interest. Thus, in a particular embodiment, expression of IL-2 comprises the cells of the tissue or organ of interest, such as those cells which make up said tissue or organ (e.g. neurones), expressing IL-2. In some embodiments, expression of IL-2 comprises neurons, oligodendrocytes and/or astrocytes. In one embodiment, expression of IL-2 comprises astrocytes. The expression of IL-2 by/in astrocytes will be appreciated to provide several advantages: 1) astrocytes are efficient secretory cells which are widely distributed across the brain; 2) astrocytes are well represented in the spinal cord, providing the possibility of administration or expression of IL-2 in the spinal cord; 3) astrocytes demonstrate temporal and spatial numerical increases during neuroinflammatory events such as traumatic brain injury; and 4) expression of the astrocyte-specific promoter GFAP is upregulated in response to injury and disease (FIG. 5B). In a further embodiment, expression of IL-2 comprises expression in cells other than the regulatory T cells which make up the expanded population of regulatory T cells produced according to the methods defined herein. Thus, in a yet further embodiment, expression of IL-2 is not in a population of regulatory T cells produced according to the methods defined herein. In one embodiment, administration or expression of IL-2 comprises expression from the endogenous IL-2-encoding gene of cells of the tissue or organ of interest. According to this embodiment, expression of IL-2 in the cells of the tissue or organ does not comprise transfection, transduction or introduction of exogenous sequence. Thus, in one embodiment, expression of IL-2 in the cells of the tissue or organ comprises tissue- or organ-specific stimulation using a compound which upregulates or “turns on” expression of the gene encoding for IL-2 only in those cells of the tissue or organ of interest. It will be appreciated that, according to this embodiment, stimulation of expression of the endogenous gene encoding IL-2 is specific and localised only to the tissue or organ of interest.

In an alternative embodiment, administration or expression of IL-2 comprises introducing into the cells of the tissue or organ exogenous sequence encoding IL-2. Thus, in one embodiment, administration or expression of IL-2 comprises expression from an exogenous sequence. In a further embodiment, administration or expression of IL-2 comprises expression from a transgene. In a yet further embodiment, the transgene comprises a gene or an element encoding for IL-2. In a particular embodiment, the exogenous sequence is an IL-2 encoding sequence. In a further embodiment, the transgene comprises an IL-2 encoding sequence or gene.

In one embodiment, the exogenous sequence encoding IL-2 is in the form of a transgene comprising a tissue- or organ-specific promoter. Such tissue- or organ-specific promoters are known in the art and include promoters which drive the expression of tissue- or organ-specific genes. In one embodiment, the transgene comprises a tissue- or organ-specific promoter which specifically drives expression in the tissue or organ of interest. In a further embodiment, the transgene comprises a tissue- or organ-specific promoter which does not lead to expression in a tissue or organ other than the tissue or organ of interest. Thus, in one embodiment, the transgene comprises a promoter which drives expression specifically in neurones. In a further embodiment, the transgene comprises a promoter which drives expression specifically in cells of the central and/or peripheral nervous system. In a yet further embodiment, the transgene comprises a promoter which drives expression in the central nervous system but not in the peripheral nervous system. In another embodiment, the transgene comprises a promoter which drives expression in the peripheral nervous system but not in the central nervous system. In one embodiment, the transgene comprises a promoter which drives expression specifically in the brain. In a particular embodiment, the transgene comprises a promoter which drives expression specifically in astrocytes. In a further embodiment, the transgene comprises a GFAP promoter. In a yet further embodiment, the transgene comprises a minimal GFAP promoter.

In a further embodiment, administration or expression of IL-2 comprises a transgene which comprises an element which promotes or induces the expression of IL-2 in the presence of an exogenous compound. Such elements which promote or induce expression are known in the art and include, for example, tetracycline (Tet)-inducible systems. Tet-inducible systems provide reversible control of transcription and utilise a tetracycline-controlled transactivator (tTA) which binds tetracycline operator (TetO) sequences contained in a tetracycline response element (TRE) placed upstream of the gene/coding region of interest (and its promoter, such as a tissue-specific promoter). They may either be TetOff or TetOn systems. The TetOff system of inducible expression (also known as the tTA-dependent system) uses a tTA protein created by fusing the tetracycline repressor (TetR), found in Escherichia coli bacteria, with the activation domain of another protein, VP16, found in the Herpes Simplex Virus. The resulting tTA is able to bind TetO sequences within the TRE in the absence of tetracycline and promote expression of the downstream gene/coding region. In the presence of tetracycline, tTA binding to the TetO sequences is prevented, resulting in reduced gene expression. Conversely, the TetOn system (also known as the rtTA-dependent system) uses a reverse Tet repressor (rTetR) to create a reverse tetracycline-controlled transactivator (rtTA) protein which relies on the presence of tetracycline to promote expression. Therefore, rtTA only binds to TetO sequences within the TRE and promotes expression in the presence of tetracycline. Specific examples of TetOn systems include, but are not limited to, TetOn Advanced, TetOn 3G and the T-REx system from Life Technologies. Derivatives and analogues of tetracycline may be used with either the TetOff or TetOn systems and include, without limitation, doxycycline and minocycline (e.g. minomycin). Such derivatives/analogues will be appreciated to provide significant advantages compared to tetracycline such as increased stability in the case of doxycycline and/or the ability to cross the blood-brain barrier in the case of minocycline (Chtarto et al. 2003, doi: https://doi.org/10.1016/j.neulet.2003.08.067). Thus, in certain embodiments, the exogenous sequence encoding IL-2, such as the transgene comprising a tissue- or organ-specific promoter, further comprises a tetracycline response element (TRE). As such, in one embodiment, administration or expression of IL-2 is tetracycline-dependent or tetracycline-inducible. In a further embodiment, administration or expression of IL-2 comprises introducing into the cells of the tissue or organ exogenous sequence encoding a reverse tetracycline-controlled transactivator (rtTA). In one embodiment, the exogenous sequence encoding an rtTA comprises a tissue- or organ-specific promoter, i.e. expression of the rtTA-encoding sequence is under the control of a tissue- or organ-specific promoter as disclosed herein. Thus, in a further embodiment, the exogenous sequence encoding an rtTA comprises a promoter specific for the nervous system, such as the central nervous system (e.g. the brain). In a yet further embodiment, expression of the rtTA-encoding sequence is under the control of a promoter specific for the nervous system, such as the central nervous system (e.g. the brain). In a particular embodiment, the exogenous sequence encoding an rtTA comprises a promoter which drives expression specifically in astrocytes, such as a GFAP promoter or a minimal GFAP promoter. Such an rtTA-encoding exogenous sequence may be a separate sequence to the exogenous sequence encoding IL-2, e.g. it may be separate from the IL-2 transgene comprising a tissue- or organ-specific promoter. Alternatively, such an rtTA-encoding exogenous sequence may be comprised together with the IL-2-encoding sequence, e.g. it may be comprised in the same transgene. Thus, in some embodiments, administration or expression of IL-2 comprises a TetOn system. It will therefore be appreciated that, in one embodiment, administration or expression of IL-2 comprises the administration of tetracycline or a derivative/analogue of tetracycline, such as doxycycline or minocycline. In a particular embodiment, administration or expression of IL-2 comprises administration of minocycline, such as administration of minomycin.

The use of tetracycline-dependent or tetracycline-inducible administration or expression of IL-2 provides another level of control and allows the administration or expression of IL-2 to be ‘switched’ on or off. Such switching will be appreciated to be advantageous in the methods described herein by allowing the expansion of a population of regulatory T cells in a tissue or organ to be temporally controlled. For example, expression of IL-2 may be switched ‘on’ by administering tetracycline or a derivative/analogue thereof when inflammation of the central and/or peripheral nervous system, such as neuroinflammation and/or inflammation of the brain, is detected/diagnosed. Alternatively, expression of IL-2 may be switched ‘on’ following an acute injury to the brain or head, such as traumatic brain injury or stroke. Expression of IL-2 may then be switched ‘off’ by removal of tetracycline or a derivative/analogue thereof when inflammation, such as neuroinflammation, is no longer detected or has reduced. Expression may also be switched ‘off’ after the subject is deemed to no longer be at risk of an acute brain injury, such as traumatic brain injury or stroke. Said use of tetracycline-dependent or tetracycline-inducible administration or expression of IL-2 further provides dose-dependent IL-2 administration of expression. For example, the level and/or amount of IL-2 administration or expression may be altered and/or titrated in the tissue or organ to depend on the level and/or amount of inflammation, such as neuroinflammation, in the tissue or organ. Therefore, expression of IL-2 may be switched ‘on’ by administering a particular dose of tetracycline or a derivative/analogue thereof when inflammation of the central and/or peripheral nervous system, such as neuroinflammation and/or inflammation of the brain, is detected/diagnosed and said dose may be increased if the inflammation persists. Similarly, said dose may be decreased if the inflammation decreases following initial administration of tetracycline or a derivative/analogue thereof.

In another embodiment, administration or expression of IL-2 comprises a transgene which comprises an element which prevents the expression of IL-2. Such element which prevents expression may be removed and/or deactivated in cells of the tissue or organ of interest. In certain embodiments, there is no removal or deactivation of the element which prevents expression in cells other than those of the tissue or organ of interest. Thus, in one embodiment, removal or deactivation of the element which prevents expression does not occur in a population of regulatory T cells produced according to the methods defined herein. In a further embodiment, the element which prevents expression is a stop cassette. In one embodiment, said stop cassette is comprised in the transgene as defined herein and is situated upstream of the gene encoding for IL-2. In a further embodiment, said stop cassette is flanked by sites which are recognised by a recombinase enzyme. Such recombinase enzymes include Cre recombinase and Flp recombinase and are capable of recognising and recombining sites such as LoxP and FRT, respectively. Recombination of said sites results in removal, deletion and/or inactivation of the sequence comprised between them. Thus, in one embodiment, the stop cassette is flanked by LoxP recombination sites. According to this embodiment, cells of the tissue or organ of interest may express the Cre recombinase in order to recombine the recombination sites in said cells. In a particular embodiment, said expression of Cre recombinase is localised to, specifically in or only in cells of the tissue or organ of interest. Such localised or specific expression of Cre recombinase in cells of the tissue or organ of interest may be driven by methods as defined herein using a tissue- or organ-specific promoter, or may be by any other method known in the art. Such methods may include tissue- or organ-specific delivery of Cre recombinase enzyme and tissue- or organ-specific delivery of Cre recombinase encoding sequence, such as tissue- or organ-specific delivery of Cre recombinase encoding mRNA or a Cre recombinase encoding transgene. Thus, in certain embodiments, localised or specific expression of Cre recombinase is driven by a tissue- or organ-specific promoter. In one embodiment, localised or specific expression of Cre recombinase is driven by a promoter which drives expression specifically in neurones. In a further embodiment, localised or specific expression of Cre recombinase is driven by a promoter which drives expression specifically in cells of the central and/or peripheral nervous system. In a yet further embodiment, localised or specific expression of Cre recombinase is driven by a promoter which drives expression in the central nervous system but not in the peripheral nervous system. In another embodiment, localised or specific expression of Cre recombinase is driven by a promoter which drives expression in the peripheral nervous system but not in the central nervous system. In one embodiment, localised or specific expression of Cre recombinase is driven by a promoter which drives expression specifically in the brain. In a particular embodiment, localised or specific expression of Cre recombinase is driven by a promoter which drives expression specifically in astrocytes. In a further embodiment, localised or specific expression of Cre recombinase is driven by a PLP promoter. In another embodiment, localised or specific expression of Cre recombinase is driven by a CaMKIIa promoter.

It will be appreciated that, according to embodiments wherein an element which prevents the expression in cells other than those of the tissue or organ of interest is utilised, the presence of a tissue- or organ-specific promoter to control expression of IL-2 may not be required. Thus, in one embodiment, the transgene comprising an element which prevents expression in cells other than those of the tissue or organ of interest does not comprise a tissue- or organ-specific promoter. In another embodiment, the transgene comprising an element which prevents expression in cells other than those of the tissue or organ of interest further comprises a tissue or organ-specific promoter. In such an embodiment, expression of IL-2 will be subject to a further level of control to further ensure tissue- or organ-specific administration or expression.

In one embodiment, the transgene as defined herein is introduced into the cells of the tissue or organ of interest by transduction, such as transduction using a virus or viral vector. In a particular embodiment, the transduction uses an adeno-associated virus. Thus, in one embodiment, administration of IL-2 comprises transduction, such as viral transduction. In a further embodiment, administration of IL-2 comprises adeno-associated virus transduction.

In one embodiment, transduction of the transgene as defined herein utilises a viral vector which specifically targets or infects the cells of the tissue or organ of interest. Thus, in one embodiment, transduction of the transgene as defined herein specifically targets or infects the cells of the tissue or organ of interest. According to this embodiment, it will be appreciated that transduction using a viral vector of the transgene as defined herein does not target or infect a population of regulatory T cells. In a further embodiment, transduction of the transgene as defined herein comprises a viral vector which is capable of accessing the tissue or organ of interest and is capable of crossing a barrier which separates the tissue or organ of interest from other tissues, organs or the rest of the organism. Thus, in one embodiment, transduction comprises a viral vector capable of specifically targeting or infecting the nervous system. In a further embodiment, transduction comprises a viral vector capable of targeting or infecting the central nervous system. In an alternative embodiment, transduction comprises a viral vector capable of targeting or infecting the peripheral nervous system. In a yet further embodiment, transduction comprises a viral vector capable of targeting or infecting the brain.

In a particular embodiment, transduction comprises a viral vector capable of crossing the blood-brain barrier. In one embodiment, transduction comprises a blood-brain barrier-crossing adeno-associated virus. Thus, in one embodiment, transduction comprises a neurotropic virus or viral vector. In another embodiment, the viral vector is a neurotropic virus or viral vector. Examples of neurotropic viruses and viral vectors capable of crossing the blood-brain barrier include, but are not limited to, AAVrh.8, AAVrh10 and AAV9 as well as its variants and derivatives (e.g. AAVhu68 and PHP.B). In certain embodiments, the transgene as defined herein is comprised in a viral vector, such as a neurotropic virus or viral vector and/or an adeno-associated virus vector. In a further embodiment, transduction comprises the adeno-associated virus variant AAV9 and its derivatives, such as PHP.B. In a yet further embodiment, transduction comprises a PHP.B viral vector. In another embodiment, the transgene as defined herein is comprised in a PHP.B viral vector. Thus, in one embodiment, the transduction and/or the viral vector comprises PHP.B-GFAP-IL2, which is the PHP.B derivative of AAV9 comprising a transgene which contains an IL-2 encoding sequence and the astrocyte-specific promoter, GFAP. Viral vectors may be used to integrate the target sequence, such as a transgene, into the host cell genome, such as the genome of a cell of the tissue or organ of interest. Thus, in certain embodiments, transduction comprises integration of the transgene as defined herein into the genome of a cell of the tissue or organ of interest such that long-term expression of the transgene in the tissue or organ is achieved. Viral vectors, such as neurotropic viruses or viral vectors and adeno-associated viral vectors, may also be used to enable stable or long-term expression without integration of the target sequence into the host cell genome. Thus, in one embodiment, the transgene and/or target sequence are stably maintained outside the host cell genome.

References herein to a “virus” and/or “viral vector” include a virus which is non-lytic or lysogenic. Such viruses will be appreciated to achieve infection of a cell, such as a cell of the tissue or organ of interest, or introduction of a transgene into a cell without death or destruction of said cell.

It will be appreciated from the disclosures presented herein that combination of a virus or viral vector which specifically targets or infects cells of the tissue- or organ of interest (e.g. a neurotropic virus or viral vector) and a promoter which drives expression specifically in cells of the tissue or organ of interest, provides exceptional specificity. Such specificity provides a so-called ‘dual lock’, restricting both the cells into which the transgene is targeted or infected and in which cells the transgene is expressed. Thus, in one embodiment, the combination of a tissue- or organ-specific viral vector and tissue- or organ-specific promoter as defined herein provides that only those cells of the tissue or organ of interest comprise the transgene as defined herein and only those cells of the tissue or organ of interest are capable of expressing said transgene. In a further embodiment, the combination of a tissue- or organ-specific viral vector and tissue- or organ-specific promoter as defined herein provides that only those cells of the tissue or organ of interest comprise an IL-2-encoding gene and only those cells of the tissue or organ of interest are capable of expressing said gene.

In a yet further embodiment, the combination of a tissue- or organ-specific viral vector and tissue- or organ-specific promoter as defined herein together with an inducible element, such as a tetracycline-inducible element, provides that only those cells of the tissue or organ of interest comprise the transgene as defined herein and only those cells of the tissue or organ of interest are capable of expressing said transgene when an activator of the inducible element is administered (e.g. tetracycline, doxycycline or minocycline/minomycin). In one embodiment, the combination of a tissue- or organ-specific viral vector and tissue- or organ-specific promoter as defined herein together with an inducible element, such as a tetracycline-inducible element, provides that only those cells of the tissue or organ of interest comprise an IL-2-encoding gene and only those cells of the tissue or organ of interest are capable of expressing said gene when an activator of the inducible element is administered (e.g. tetracycline, doxycycline or minocycline/minomycin). In a further embodiment, said combination provides that only those cells of the tissue or organ of interest comprise an inducible IL-2-encoding gene and only those cells of the tissue or organ of interest are capable of expressing a reverse tetracycline-controlled transactivator (rtTA) which leads to the expression of IL-2 when an activator of the inducible element is administered (e.g. tetracycline, doxycycline or minocycline/minomycin).

Administration of IL-2 as defined herein may further comprise administration of IL-2 directly to the tissue or organ of interest. Examples of direct administration include injection directly into the tissue or organ of interest, such as by intracranial injection, or utilise a suitable delivery device. Such delivery devices are known in the art and, according to the present disclosures, allow for the controlled and/or sustained administration of IL-2 for the duration of treatment (e.g. chronically or for duration of treatment of an acute inflammatory disease or disorder).

The duration of IL-2 administration as defined herein can be altered to depend on the treatment and the characteristics of the particular inflammatory condition or disease to be treated by the methods described herein. For example, administration of IL-2 may be chronic. Alternatively, administration of IL-2 may be for the duration of treatment for the disease or disorder, such as in the treatment of an acute inflammatory condition or traumatic injury. Thus, in certain embodiments, the duration of administration or expression of IL-2 depends on the disease or disorder to be treated or on the duration of the treatment. In one embodiment, administration or expression of IL-2 is acute.

It will be appreciated that IL-2 and a targeting moiety specific for a tissue or organ may be combined or co-administered. Therefore, the administration of IL-2 may comprise expression of IL-2 in the tissue or organ of interest as defined herein (e.g. tissue- or organ-specific expression) and can be combined with a targeting moiety specific for the tissue or organ of the subject. Furthermore, administration of IL-2 may comprise administration of IL-2 in protein or peptide form and can be combined with a targeting moiety specific for the tissue or organ of the subject.

References herein to the term “targeting moiety” refer to any moiety that provides for the tissue- or organ-specific administration or expression of IL-2 as defined herein. Furthermore, said targeting moiety will be appreciated to provide for the localised administration or expression of IL-2 as defined herein.

Thus, in one embodiment of the present invention, the methods defined herein comprise administration of a targeting moiety specific for the tissue or organ of the subject. In a further embodiment, the targeting moiety specific for the tissue or organ of the subject localises IL-2 in or to the tissue or organ of interest. Thus, in one embodiment, the targeting moiety specific for the tissue or organ of the subject localises IL-2 only in or to the tissue or organ of interest. In a further embodiment, the targeting moiety specific for the tissue or organ of the subject prevents localisation of IL-2 to other tissues or organs other than the tissue or organ of interest, or localises IL-2 away from tissues or organs other than the tissue or organ of interest. In another embodiment, the targeting moiety provides for expression of IL-2 in the tissue or organ of interest. Thus, in one embodiment, the targeting moiety specific for the tissue or organ of the subject provides for expression of IL-2 only in the tissue or organ of interest. Such references herein to “in the tissue or organ of interest” further include wherein said effect is in the cells which make up said tissue or organ (e.g. neurones and/or astrocytes).

In one embodiment, the targeting moiety specific for the tissue or organ of the subject is a virus or viral vector as defined herein. In a further embodiment, said virus or viral vector specifically targets or infects the tissue or organ of interest or specifically targets or infects cells of the tissue or organ of interest. Thus, according to this embodiment, said targeting moiety specific for the tissue or organ of interest which is a virus or viral vector that does not target or infect cells in other tissues or organs other than the tissue or organ of interest, or target or infect cells which make up a tissue or organ other than the tissue or organ of interest. Also according to this embodiment, it will be appreciated that said targeting moiety specific for the tissue or organ as defined herein does not target or infect a population of regulatory T cells. In a further embodiment, the targeting moiety specific for the tissue or organ of a subject as defined herein comprises a virus or viral vector which is capable of accessing the tissue or organ of interest and is capable of crossing a barrier which separates the tissue or organ of interest from other tissues, organs or the rest of the subject. Thus, in one embodiment, the targeting moiety specific for a tissue or organ comprises a virus or viral vector capable of specifically targeting or infecting the nervous system, such as a neurotropic virus or viral vector. In a further embodiment, the targeting moiety specific for a tissue or organ comprises a virus or viral vector capable of targeting or infecting the central nervous system. In an alternative embodiment, the targeting moiety specific for a tissue or organ comprises a virus or viral vector capable of targeting or infecting the peripheral nervous system.

In a particular embodiment, the targeting moiety specific for a tissue or organ comprises a virus or viral vector capable of crossing the blood-brain barrier. In one embodiment, the targeting moiety specific for a tissue or organ comprises a blood-brain barrier-crossing adeno-associated virus. Thus, in certain embodiments, the targeting moiety specific for a tissue or organ comprises a neurotropic virus or viral vector. In one embodiment, the targeting moiety is selected from a neurotropic virus or viral vector, such as AAVrh.8, AAVrh10 or AAV9 and variants and derivatives (e.g. AAVhu68 and PHP.B). In a further embodiment, the targeting moiety specific for a tissue or organ comprises the adeno-associated virus variant PHP.B. In certain embodiments, the transgene as defined herein is comprised in a targeting moiety specific for a tissue or organ, such as an adeno-associated virus vector, which is comprised within an adeno-associated virus as defined herein. In one embodiment, the transgene as defined herein is comprised in a neurotropic virus or viral vector, such as a PHP.B viral vector. Thus, in a further embodiment, the transgene which contains an IL-2 encoding sequence and the astrocyte-specific promoter, GFAP or minimal GFAP, is comprised in the AAV9 derivative PHP.B virus/viral vector and the virus/viral vector is PHP.B-GFAP-IL2.

According to a further aspect of the invention, there is provided a method for the expansion of a population of regulatory T cells in a tissue or organ in vivo. Embodiments of the present aspect will be appreciated to be equivalent and comparable to all embodiments previously described herein. Thus, in certain embodiments, the term “of a subject” as described herein is synonymous with “in vivo”.

In one embodiment, the method for expanding a population of regulatory T cells in a tissue or organ in vivo comprises administration of IL-2 as described herein. In a further embodiment, the method for expanding a population of regulatory T cells in a tissue or organ in vivo comprises administration of a targeting moiety specific for the tissue or organ of a subject in vivo. In one embodiment, the administration of IL-2, which may comprise expression of IL-2, is combined with a targeting moiety specific for a tissue or organ in vivo. In a further embodiment, the method for expanding a population of regulatory T cells in a tissue or organ in vivo comprises a virus or viral vector which comprises an IL-2-encoding gene. In one embodiment, said virus or viral vector is capable of targeting or infecting a tissue or organ of interest. In a particular embodiment, said virus or viral vector capable of targeting or infecting a tissue or organ of interest, specifically targets or infects cells of a tissue or organ of interest. In a further embodiment, the method for expanding a population of regulatory T cells in a tissue or organ in vivo comprises a virus or viral vector which comprises a tissue- or organ-specific promoter. Thus, in a particular embodiment, the method for expanding a population of regulatory T cells in a tissue or organ in vivo comprises administration of a targeting moiety specific for the tissue or organ of interest, wherein said targeting moiety is a virus or viral vector which crosses the blood-brain barrier as defined herein. In a further embodiment, the method for expanding a population of regulatory T cells in a tissue or organ in vivo comprises administration of a targeting moiety specific for the tissue or organ of interest, wherein said targeting moiety is specific for the nervous system such as the central and/or peripheral nervous system. In a yet further embodiment, the targeting moiety specific for a tissue or organ of interest is specific for astrocytes. In another embodiment, the method for expanding a population of regulatory T cells in a tissue or organ in vivo comprises administration of a neurotropic virus or viral vector containing the transgene as defined herein, such as administration of PHP.B-GFAP-IL2.

According to one aspect of the invention, there is provided a population of regulatory T cells expanded according to or obtained by the methods described herein. Thus, in one embodiment, there is provided an expanded population of regulatory T cells which have been expanded in a tissue or organ of a subject by administration of IL-2 and a targeting moiety specific for said tissue or organ.

Pharmaceutical Compositions

According to one aspect of the invention, there is provided a pharmaceutical composition comprising IL-2 and a targeting moiety specific for a tissue or organ of a subject, wherein said targeting moiety is specific for the central and/or peripheral nervous system.

In one embodiment, the pharmaceutical composition comprises IL-2 which promotes the expansion of a population of regulatory T cells. In a yet further embodiment, the pharmaceutical composition comprises a targeting moiety specific for a tissue or organ of a subject. In one embodiment, the targeting moiety specific for a tissue or organ of a subject is a virus or viral vector which specifically targets or infects cells of the tissue or organ and drives tissue- or organ-specific expression of IL-2 as described herein. Thus, according to this aspect of the invention, there is provided a pharmaceutical composition comprising a tissue- or organ-specific viral vector which expands a population of regulatory T cells in said tissue or organ of the subject. In particular embodiments, the pharmaceutical composition expands a population of regulatory T cells specifically or locally in a tissue or organ of interest in a subject.

In one embodiment, the pharmaceutical composition as defined herein comprises a targeting moiety capable of crossing a barrier which separates a tissue or organ of interest from other tissues or organs or from the rest of the organism. Thus, in one embodiment, the pharmaceutical composition as defined herein comprises a blood-brain barrier crossing virus or viral vector, such as an adeno-associated virus and/or a neurotropic virus or viral vector. In a further embodiment, the pharmaceutical composition as defined herein comprises the adeno-associated virus variant AAV9 or its derivatives, such as PHP.B. In a further embodiment, the viral vector comprised in the pharmaceutical composition as defined herein comprises a gene, such as a transgene, which encodes for IL-2. In a yet further embodiment, the transgene comprised in the viral vector of the pharmaceutical composition further comprises a tissue- or organ-specific promoter as defined herein.

Thus, in certain embodiments, the pharmaceutical composition as defined herein comprises a tissue- or organ-specific virus or viral vector capable of targeting or infecting cells of the tissue or organ of interest, comprising an IL-2-encoding gene, expression of which is driven by a tissue- or organ-specific promoter. In one particular embodiment, the pharmaceutical composition as defined herein comprises a viral vector, such as an adeno-associated virus (e.g. AAV9 or its derivatives, such as PHP.B), which specifically targets or infects neurones or the nervous system, such as the brain, (i.e. a neurotropic virus or viral vector) which comprises an IL-2-encoding gene, expression of which is driven by a tissue- or organ-specific promoter. In a further embodiment, the pharmaceutical composition as defined herein comprises the adeno-associated virus AAV9, which comprises an IL-2-encoding gene, expression of which is driven locally in a neurone/astrocyte or in the nervous system by a GFAP promoter or a minimal GFAP promoter. In a yet further embodiment, the adeno-associated virus is a derivative of AAV9, such as PHP.B. Thus, in one embodiment, the pharmaceutical composition comprises PHP.B-GFAP-IL2.

According to some embodiments, the pharmaceutical composition, in addition to a tissue- or organ-specific virus or viral vector as defined herein, further comprises one or more pharmaceutically acceptable excipients.

Generally, the present pharmaceutical compositions will be utilised with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a composition comprising the targeting moiety specific for a tissue or organ as defined herein in a discrete location (e.g. within a tissue or organ of interest), may be chosen from thickeners such as carboxymethylcellulose, polyvinylpyrrolidone, gelatine and alginates. Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16^{th} Edition).

Therapeutic Uses and Methods

It will be appreciated from the disclosures presented herein that the method of expanding a population of regulatory T cells, pharmaceutical compositions and methods of treatment of the present invention will find particular utility in the treatment and/or amelioration of diseases or disorders mediated by inflammation and/or in the reduction of inflammation. It will be further appreciated that a population of regulatory T cells expanded according to the methods and disclosures presented herein will also find utility in the treatment and/or amelioration of diseases or disorders mediated by inflammation and/or in the reduction of inflammation.

Thus, according to one aspect of the invention, there is provided a method for expanding a population of regulatory T cells in a tissue or organ of a subject for use in the treatment and/or amelioration of a disease or disorder mediated by inflammation, wherein said tissue or organ is the central and/or peripheral nervous system. In another aspect of the invention, there is provided a method for expanding a population of regulatory T cells in a tissue or organ of a subject for use in the reduction of inflammation, wherein said tissue or organ is the central and/or peripheral nervous system. In a further aspect of the invention, there is provided a method for expanding a population of regulatory T cells in a tissue or organ of a subject for use in the treatment and/or amelioration of an autoimmune disease, wherein said tissue or organ is the central and/or peripheral nervous system.

In another aspect of the invention, there is provided a population of expanded regulatory T cells in a tissue or organ of a subject produced according to the methods defined herein for use in the treatment and/or amelioration of a disease or disorder mediated by inflammation or for use in the reduction of inflammation. Such diseases or disorders may include inflammatory conditions, autoimmune diseases and/or diseases associated with transplant, such as transplant rejection or graft vs. host disease. In one embodiment, the expanded population of regulatory T cells in a tissue or organ of a subject produced according to the methods defined herein has been expanded by administration of IL-2 and a targeting moiety specific for said tissue or organ. In a further embodiment, the population of expanded regulatory T cells in a tissue or organ of a subject produced according to the methods defined herein has been expanded by tissue- or organ-specific expression of IL-2 as defined herein. In another embodiment, the population of expanded regulatory T cells in a tissue or organ of a subject has been expanded by tissue- or organ-specific expression of IL-2 promoted or induced by an inducible element, such as a tetracycline-inducible element. In a yet further embodiment, the population of expanded regulatory T cells in a tissue or organ of a subject produced according to the methods defined herein is for use in the treatment and/or amelioration of a disease or disorder of the nervous system. In one embodiment, the population of expanded regulatory T cells in a tissue or organ of a subject produced according to the methods defined herein is for use in the treatment and/or amelioration of the central and/or peripheral nervous system. In a yet further embodiment, the population of expanded regulatory T cells in a tissue or organ of a subject produced according to the methods defined herein is for use in the treatment and/or amelioration of neuroinflammation. In certain embodiments, the population of expanded regulatory T cells in a tissue or organ of a subject produced according to the methods defined herein is for use in the treatment and/or amelioration of inflammation in the brain. Thus, according to one embodiment, the inflammation as defined herein is inflammation of the brain. In a further embodiment, inflammation of the brain is due to an injury of the brain or head, such as traumatic brain injury or stroke. In another embodiment, the population of expanded regulatory T cells in a tissue or organ of a subject produced according to the methods defined herein is for use in the treatment and/or amelioration of a neurological disease or disorder. Thus, in one embodiment, the inflammation in the brain is due to a neurological disease or disorder, such as a traumatic neurological disease or disorder. In another embodiment, the population of expanded regulatory T cells in a tissue or organ of a subject produced according to the methods defined herein is for use in the treatment and/or amelioration of cognitive impairment, such as cognitive impairment caused by neuroinflammation. In one embodiment, the population of expanded regulatory T cells in a tissue or organ is for use in the reduction of cognitive impairment. In a further embodiment, the inflammation in the brain is due to an acute traumatic injury, disease or disorder. Thus, in a further embodiment, the neurological disease or disorder is other than (i.e. is not) a neurodegenerative disease or disorder, such as Alzheimer's and/or Parkinson's disease. Another example is an autoimmune disease or disorder and/or wherein the inflammation is due to an autoimmune disease or disorder.

According to a further aspect of the invention, there is provided a method of treating a disease or disorder mediated by inflammation and/or for the reduction of inflammation, wherein said method either comprises a method as defined herein or administering to a subject in need thereof a pharmaceutical composition comprising IL-2 and a targeting moiety specific for a tissue or organ of a subject as defined herein. In one embodiment, said method of treatment comprises administering a virus or viral vector comprising a gene encoding IL-2 as defined herein to a subject in need thereof. In one embodiment, the method of treatment as defined herein, comprises administering to a subject in need thereof a virus or viral vector which specifically targets or infects a tissue or organ affected by a disease or disorder mediated by inflammation or affected by inflammation. In certain embodiments, the method of treatment as defined herein, further comprises administering to a subject in need thereof a virus or viral vector comprising a gene encoding IL-2, expression of which is driven by a tissue- or organ-specific promoter. In a further embodiment, the method of treatment as defined herein comprises administering to a subject in need thereof a virus or viral vector comprising a gene encoding IL-2, expression of which is driven by a tissue- or organ-specific promoter and an inducible element, such as a tetracycline-inducible element. In an alternative embodiment, the method of treatment comprises administering to a subject a virus or viral vector comprising a gene encoding IL-2, expression of which is driven by an inducible element, such as a tetracycline-inducible element, under the control of a tissue- or organ-specific promoter. In further embodiments, the method of treatment as defined herein comprises administering to a subject in need thereof a neurotropic virus comprising a gene encoding IL-2, expression of which is driven by a tissue- or organ-specific promoter, such as administering PHP.B-GFAP-IL2.

In certain embodiments, said subject in need thereof is suffering from a disease or disorder mediated by inflammation. In further embodiments, the subject in need thereof is suffering from inflammation. In yet further embodiments, the subject in need thereof is suffering from an autoimmune disease or disorder. In one embodiment, said disease or disorder is a disease or disorder of the nervous system, such as the central and/or peripheral nervous system. In a further embodiment, said disease or disorder is a disease or disorder of the brain. In yet further embodiments, said disease or disorder is a neurological disease or disorder other than (i.e. is not) a neurodegenerative disease or disorder, such as Alzheimer's disease or Parkinson's disease. In another embodiment, said inflammation is neuroinflammation, such as inflammation of the brain. In one embodiment, said inflammation is inflammation of the brain due to an injury of the brain or head, such as traumatic brain injury or stroke. Thus, in some embodiments, said inflammation is inflammation of the brain due to an acute traumatic injury.

### EXAMPLES

### Example 1: Regulatory T Cells are Present in the Parenchyma of the Healthy Mouse Brain

To investigate the presence of regulatory T cells in the brain, a tissue traditionally thought to be isolated from the immune system, tissue from mouse brain parenchyma, perivascular space and intravascular regions were prepared for confocal microscopy and immunostained for CD4 (a marker of T cells) and FoxP3 (a specific marker for regulatory T cells). These tissues were further stained with fluorescent-labelled lectin to label the vasculature and DAPI to identify cell nuclei. Representative images are shown in FIG. 1A and magnifications and 3D-reconstructions are shown in FIG. 1B. FIG. 1C shows the numbers of regulatory T cells in the perfused mouse brain as determined by flow cytometry.

As can be seen from the data, regulatory T cells can be readily identified in the brain of healthy mice by both microscopic and flow cytometric analysis. Depending on the age of the mice analysed, the numbers of regulatory T cells detectable in the brain ranged from approximately 100 to over 2,000 cells, with the majority of mice comprising approximately 100-1,000 regulatory T cells in the brain.

### Example 2: Brain-Resident Regulatory T Cells Acquire a Residency Phenotype In Situ During a Prolonged Brain Transit

Parabiosis experiments were performed to determine if regulatory T cells seed the brain from the periphery and whether they are capable of acquiring a resident-like phenotype. Parabiosis pairs were established using CD45.1+ and CD45.2+ mice and samples from the brain of each mouse taken at 2, 4, 8 and 12 weeks (FIG. 2A). As can be seen, both CD69+ and CD69− regulatory T cells which have been derived from the donor mouse can be identified in the brain and blood (FIG. 2B). The proportion of regulatory T cells present in the brain and blood which were derived from the donor mouse (determined using CD45.1 or CD45.2 expression) was measured and their phenotype determined (FIGS. 2C and 2D).

As is demonstrated by the data and population flow diagram generated from said data (FIG. 2E), regulatory T cells seed the brain from the periphery and can be detected as being derived from a parabiotic donor mouse. Donor-derived regulatory T cells in the brain display a tissue resident phenotype, showing that this can be acquired during brain transit. However, the data demonstrate that the naïve regulatory T cell population, which is disproportionately increased by IL-2 administration, seeds the brain at approximately 10-fold lower efficiency than activated regulatory T cells (FIG. 2E). Thus, there is a need to expand regulatory T cells specifically in the brain without significantly expanding the peripheral regulatory T cell population.

### Example 3: Transgenic Mouse Model for Proof-of-Principle Brain-Specific Regulatory T Cell Expansion

In order to test the principle of using brain-specific IL-2 delivery to expand the regulatory T cell population specifically in the brain transgenic mouse models were developed. The Rosa^{fl-Stop-fl}IL-2 mice were developed, where IL-2 expression is switched on with Cre-activity under a weak constitutive promoter. Expression in this system is approx. 4-fold lower per cell than endogenous expression in IL-2-producing cells (FIG. 3A). The system therefore operates through altered localisation of expression, rather than over-expression. To test this system in the brain, two brain specific Cre lines were used to induce restricted expression, the PLP-Cre ER^{T} (FIG. 3B) and the CaMKIIa Cre ER^{T} (FIG. 3C). Activation of IL-2 production through either transgene expanded the regulatory T cell population in the brain (FIGS. 3D and 3E). PLP-Cre resulted in a small increase in the periphery, while CaMKIIa Cre resulted in no peripheral increase (FIGS. 3D and 3E). Therefore, use of the CaMKIIa Cre driver was chosen for subsequent experiments. Single cell RNA-seq was performed on the brain CD4 T cells using the 10× genomics Chromium platform. The expanded brain regulatory T cells from the brains of IL-2 aCaMKII Cre mice clustered tightly with the (smaller) population of brain regulatory T cells from a wildtype mouse brain (FIG. 3F). The analysis of single-cell transcriptomic data revealed that regulatory T cells in the brain of IL-2 aCaMKII Cre mice expressed known markers such as IIr2, Gata3, and Ikzf2, indicating no unusual effect of expansion on the regulatory T cell population (FIG. 3G). Analysis of expressed cytokines demonstrated the only highly-expressed cytokine from these expanded regulatory T cells was Areg, a low-affinity epidermal growth factor receptor (EGFR) ligand, shown to be involved in wound healing and tissue repair (Zaiss et al. (2015) doi: https://doi.org/10.1016/j.immuni.2015.01.020) (FIG. 3H). The expansion of brain regulatory T cells resulted in no adverse behavioural changes in IL-2 aCaMKII Cre (αCamKII^{IL2}) mice (FIG. 3I-3S).

This data demonstrates that local provision of IL-2 is capable of specifically expanding up the brain regulatory T cell population, without expanding peripheral numbers, and that the expanded regulatory T cells have preserved their classical regulatory T cell expression profile.

### Example 4: Expanded Brain Regulatory T Cells Protect Against Traumatic Brain Injury

To determine the potential of brain-specific regulatory T cell expansion in reducing neuroinflammatory damage, the effect of moderate traumatic brain injury (TBI) given by controlled cortical impact was investigated. IL-2 aCaMKII Cre (αCamKII^{IL2}) mice and littermate controls were given moderate TBI and examined at 15 days post-TBI. While wildtype mice exhibited complete cortical death at the site of cortical impact and no evidence of neuronal recovery, IL-2 aCaMKII Cre mice demonstrated greatly reduced damage at the impact site, with compensatory expansion of the hippocampus on the ipsilateral side, reduced lesion size and preservation of neuronal tissue (FIG. 4A-4D).

This data demonstrates that local delivery of IL-2 can create a local anti-inflammatory environment, capable of preventing neurological pathology, without increasing the systemic regulatory T cell burden.

### Example 5: Astrocyte Specific Expression Using a GFAP Promoter

With proof-of-principle of the efficacy of local IL-2 provision, a delivery system that could be used in a therapeutic setting was developed. Blood-brain barrier (BBB)-crossing adeno-associated viruses (AAVs) are a powerful tool for fast-track administration of CNS therapeutics, as they allow the delivery of transgenes encoding large bioactive molecules without the need for invasive surgical procedures. AAV-based vectors are the system of choice in clinical trials due to their long-term expression of transgenes and excellent safety profile. Since AAVs represent a unique opportunity for IL-2 delivery to the CNS in a clinical setting, the recently identified AAV variant, PHP.B, which has been shown to be efficient in crossing the BBB, giving high levels of transduction throughout the CNS (Rincon et al. (2018) doi: https://doi.org/10.1038/s41434-018-0005-z) was used. Here the primary concern was on off-target production of IL-2 in the periphery, so the AAV vector was coupled to a GFAP promoter, which gives long-lasting and specific expression only in astrocytes (FIG. 5A). The combination of a neurotropic virus and a brain-specific promoter gives a ‘dual lock’ on target specificity, restricting or eliminating peripheral expression of the delivered target following systemic delivery. An AAV-PHP.B virus carrying the transgene for mouse IL-2 (NG_06779.1) under control of the astrocyte-specific GFAP promoter was then generated (PHP.B-GFAP-IL2) as follows:

The classical tri-transfection method was used with subsequent vector titration performed using a qPCR-based methodology (Rincon et al. (2018), doi: https://doi.org/10.1038/s41434-018-0005-z). The mouse IL-2 coding sequence, together with 5′ and 3′ UTR (accession number BC116845) was cloned into a single stranded AAV2-derived expression cassette, containing a full-length GFAP promoter (Brenner et al. (1994) doi: https://doi.org/10.1523/JNEUROSCI.14-03-01030.1994), woodchuck hepatitis post-transcriptional regulatory element (WPRE) and bovine growth hormone polyadenylation (bGH polyA) sequence. Control vectors were prepared by swapping the IL2 coding sequence for that encoding enhanced green fluorescent protein (EGFP).

This therapeutic design allows for targeted delivery of a self-protein expressed in the physiological range. PHP.B-GFAP-IL2 injection in WT mice successfully drove a brain-specific expansion of the regulatory T cell population (FIGS. 6A, 6B and 6E) without inducing expansion in the periphery (FIGS. 6C, 6D and 6E). Brain-specific expansion of the regulatory T cell population was also PHP.B-GFAP-IL2 dose-dependent (FIG. 6F).

Taken together, this data provides evidence that the ‘dual lock’ PHP.B-mediated gene delivery of IL-2 to the brain as provided herein leads to brain-specific expansion of regulatory T cells.

Unlike classical gene therapy approaches, where efficiency of cell transduction with the viral vector is key, production of a potent secreted factor means even small numbers of transduced cells can modulate disease. Therefore, the lower dose of 1×10⁹ vector genomes of PHP.B-GFAP-IL2 was selected to test for an effect on experimental autoimmune encephalomyelitis (EAE), the gold-standard mouse model of Multiple Sclerosis. Untreated mice developed classical EAE pathology, with severe clinical symptoms (FIG. 6G) and heavy lymphocytic infiltrate into the brain (FIG. 6H). By contrast, PHP.B-GFAP-IL2 pre-treated mice were resistant to EAE, with disease severity rapidly plateauing and reversing (FIG. 6G) and the lymphocytic infiltrate being sharply curtailed (FIG. 6H). Potential mechanisms include increased AREG and IL-10 expression (FIG. 6G).

As the kinetics of EAE are amenable to testing for curative effects, EAE was induced in a cohort of mice and then treated with 1×10⁹ vector genomes of control (PHP.B-GFAP-GFP) or the ‘dual-lock’ PHP.B-GFAP-IL2 after the development of clinical manifestations (day 10). Strikingly, the protective effect of PHP.B-GFAP-IL2 was still observed, with separation of the clinical time-course by day 15 and a sharp reduction in the cumulative clinical score (FIG. 6I).

This data provides pre-clinical evidence for the ‘dual lock’ gene delivery of IL-2 to the brain as a potent therapeutic for neuroinflammatory diseases, such as Multiple Sclerosis.

### Example 6: Expansion of Regulatory T Cells in the Brain Reduces Traumatic Brain Injury Damage

To determine the potential of the PHP.B-GFAP-IL2 therapy in reducing progression or reversing damage during traumatic brain injury, 1×10⁹ vector genomes of PHP.B-GFAP-IL2, or a control PHP.B without IL-2 (PHP.B-GFAP-GFP), were administered to mice prior to traumatic brain injury.

Microscopic analysis of the brains from control PHP.B treated mice showed major surface damage to the brain at the impact site, while treatment with PHP.B-GFAP-IL2 showed a significant reduction in the size of the impact site on the brain (FIG. 7A). Histological analysis identified a preservation of the brain cortex at the impact site, with BrdU incorporation indicating regeneration (FIG. 7B). Reduced loss of neurological tissue at 14 days post-injury as shown by histology (FIGS. 7B and 7C) and MRI (FIG. 7D) was also seen. The neuroprotective effect was also observed at the behavioural level, where the poor performance of post-TBI mice on behavioural tests was completely reversed in PHP.B-GFAP-IL2-treated mice (FIGS. 7E and 7F). These effects were likely mediated through modification of the local environment, with little change observed to the inflammatory influx (FIG. 8).

Therefore, these data show the utility of brain-specific administration of IL-2 and regulatory T cell expansion in the reduction and/or reversal of damage during traumatic brain injury.

### Example 7: Expansion of Regulatory T Cells in the Brain Reduces Severity in Stroke

To extend the above findings to a second indication, two independent mouse models of stroke were used. In both photothrombotic stroke (FIGS. 9A and 9B) and ischemic stroke (FIGS. 9C and 9D), substantial reductions in severity were observed in mice treated with PHP.B-GFAP-IL2 compared to controls (PHP.B-GFAP-GFP; both administered at a dose of 1×10⁹ vector genomes).

Together, the results presented herein validate the therapeutic potential of the ‘dual-lock’ PHP.B-GFAP-IL2 system to prevent or treat neurological damage in several independent pre-clinical models of neuroinflammatory disease, without altering peripheral immunity.

### Example 8: A Small-Molecule Inducible System for Brain-Specific Regulatory T Cell Expansion

To determine the potential for temporal control of PHP.B-GFAP-IL2 therapy, wildtype mice were administered 1×10⁹ vector genomes (total dose) of PHP.B-GFAP-GFP control vector or PHP.B-GFAP-TetR-T2A-rtTA(V7/V14).TetO-IL2 (PHP.GFAP/TetO-IL2).

The PHP.B-GFAP/TetO-IL2 vector comprises a TetO sequence upstream of the IL-2-encoding gene to which a reverse tetracycline-controlled transactivator (rtTA) protein (expressed under the control of the GFAP promoter) binds and promotes expression in the presence of tetracycline, such as minocycline/minomycin. Thus, mice were gavaged daily with minomycin (50 mg/kg) or PBS control (n=4-5 mice/group) and assessed for the proportion of Tregs in the spleen or perfused brain 14 days after treatment. As can be seen in FIG. 10, the administration of minomycin to those mice which had received the PHP.GFAP/TetO-IL2 vector lead to the substantial expansion of Tregs in the brain, with no expansion in the periphery (spleen).

Therefore, these data show that expression of IL-2 controlled by a tetracycline-inducible element expressed specifically in astrocytes through the administration of a small molecule can be used to specifically increase the regulatory T cell population in the brain, without increasing the proportion of Tregs in the periphery.

1. A method of expanding a population of regulatory T cells in a tissue or organ of a subject in need thereof, wherein said method comprises administration of IL-2 and a targeting moiety specific for said tissue or organ, and wherein said tissue or organ is the central and/or peripheral nervous system.

2. The method of claim 1, wherein the tissue or organ is the brain.

3. The method of claim 1, wherein administration of IL-2 comprises tissue- or organ-specific expression of IL-2 in said tissue or organ of said subject.

4. The method of claim 3, wherein tissue- or organ-specific expression of IL-2 is driven by a tissue- or organ-specific promoter.

5. The method of claim 1, wherein administration of IL-2 or tissue- or organ-specific expression of IL-2 in said tissue or organ comprises an exogenous IL-2 encoding sequence.

6. The method of claim 1, wherein said targeting moiety specific for the tissue or organ comprises a viral vector,
optionally wherein the viral vector is a neurotropic virus or viral vector, and optionally wherein the neurotropic virus or viral vector is an adeno-associated virus selected from AAVrh.8, AAVrh10 or AAV9 and variants and derivatives thereof.

7-8. (canceled)

9. The method of claim 6, wherein the neurotropic virus or viral vector is the adeno-associated virus variant PHP.B.

10. The method of claim 1, wherein the targeting moiety specific for the tissue or organ or the viral vector crosses a barrier which separates the tissue or organ from other tissues or organs of the subject.

11. A pharmaceutical composition comprising IL-2 and a targeting moiety specific for a tissue or organ of a subject, wherein said targeting moiety is specific for the central and/or peripheral nervous system.

12. The pharmaceutical composition of claim 11, wherein the targeting moiety specific for the tissue or organ comprises a viral vector,
optionally wherein the viral vector is a neurotropic virus or viral vector, and optionally wherein the neurotropic virus or viral vector is an adeno-associated virus selected from AAVrh.8, AAVrh10 or AAV9 and variants and derivatives thereof.

13-14. (canceled)

15. The pharmaceutical composition of claim 12, wherein the neurotropic virus or viral vector is the adeno-associated virus variant PHP.B.

16. The pharmaceutical composition of claim 11, wherein the targeting moiety specific for the tissue or organ or the viral vector crosses a barrier which separates the tissue or organ from other tissues or organs of the subject.

17. (canceled)

18. A method of treating a disease or disorder mediated by inflammation and/or for the reduction of inflammation, wherein said method comprises administering to a subject in need thereof the pharmaceutical composition according to claim 11.

19. The method according to claim 18, wherein the disease or disorder is a neurological disorder or is Multiple Sclerosis.

20. (canceled)

21. The method according to claim 18, wherein the inflammation is inflammation of the central and/or peripheral nervous system, and/or optionally wherein the inflammation is inflammation of the brain.

22. (canceled)

23. The method according to claim 18, wherein the inflammation of the brain is due to an injury to the brain or head, or wherein the inflammation of the brain is due to an acute injury to the brain or head.

24. (canceled)

25. The method according to claim 18, wherein the disease or disorder and/or inflammation is an autoimmune disease or disorder and/or the inflammation is due to autoimmunity.
